(19) United States
(12) Patent Application Publication
(10) Pub. No.: US 2018/0146866 A1
(43) Pub. Date: May 31, 2018

(54) CUTANEOUS BLOOD FLOW MONITORING DEVICE

(71) Applicant: VERISKIN, INC., San Diego, CA (US)

(72) Inventors: Mirianas CHACHISVILIS, San Diego, CA (US); Carl Frederick EDMAN, San Diego, CA (US); Eugene TU, San Diego, CA (US)

(21) Appl. No.: 15/574,126

(22) PCT Filed: May 16, 2016
(86) PCT No.: PCT/US16/32738

§ 371 Date: Nov. 14, 2017

Related U.S. Application Data
(60) Provisional application No. 62/162,597, filed on May 15, 2015.

(51) Int. Cl. A61B 5/0215; A61B 8/06; A61B 5/00; A61B 5/022; A61B 5/01

(52) U.S. Cl. CPC A61B 5/02152; A61B 8/06; A61B 5/441; A61B 5/7257; A61B 5/022; A61B 5/01; A61B 5/0095

(57) ABSTRACT

A method and a device for diagnostic of skin cancer and other mammalian skin tissue pathologies are described. The method relies on determination of pathological changes in tissue vascularization and capillary blood flow. The device uses photonic or ultrasound emitters and detectors to characterize temporal and spatial changes in blood flow associated with pulsative actions of the heart.

### CROSS-REFERENCE

This application claims the benefit of U.S. Provisional Application No. 62/162,597, filed May 15, 2015, which is incorporated herein by reference in its entirety.

### FIELD OF THE INVENTION

The field of the invention generally relates to the use of spatially resolved measurement of cutaneous blood flow, including dermal capillary flow and perfusion for use in the detection of skin cancer and other mammalian disease states.

### BACKGROUND OF THE INVENTION

Assessment of skin capillary blood refill rate has been used for determination of health status, primarily for use as an index of whole body shock or whole body dehydration. Typically, such assessment involves determining the refill time of blood vessels and capillaries of cutaneous and subcutaneous tissues following the transitory removal of blood via an applied force. Although useful as a general assessment of vascular health and vascular system function, non-invasive devices measuring tissue perfusion parameters have not been shown useful for determining or diagnosing skin disease or other pathological states in subjects.

### SUMMARY OF THE INVENTION

The disclosures described herein generally relates to a method and device for the dynamic measurement of skin blood flow parameters (e.g., capillary blood flow parameters) useful in the determination of skin disease states such as skin cancers. An exemplary form of a device comprises an approximately cylindrical member. Sensors incorporated within the member are used, for example, for measurements pertaining to the presence of blood within the region of skin against which the device is positioned, for example, by hand.

In brief, the device is configured to form contiguous contact with the skin surface by at least one outer surface of a component of the device. In one embodiment, the device is a hand held device, wherein the member is held in contact with the skin surface by a user's hand. This transitory pressure due to the periodic function of the heart results in the removal and reperfusion of blood through the skin capillaries, which may be monitored by one or more sensors of the device. In one embodiment, the device further comprises one or more sensors that may, in some instances, be contained within the member. Sensors may include photonic or ultrasonic sensors, wherein one or more sensors are configured for the determination of spatially resolved dynamic blood perfusion parameters in a spatially defined region within the skin capillary and vascular bed. In the context of the present invention, spatially resolved or spatially defined indicates that sensor data and measurements are primarily confined or otherwise restricted to blood flow, for example cutaneous blood flow, within the skin layer and not to a significant extent having blood flow measurements or data representative of deeper subcutaneous tissues, e.g. fascial layers or muscle blood flows. Devices of the present invention therefore are constructed with the purpose of conducting such spatially resolved measurements, e.g. through the selection of appropriate optical wavelength(s) and by spacing of sensing elements in the sensing head. Sensor measurements may be obtained during one or more aspects associated with the process of skin blood perfusion associated with pulsatile blood flow due to function of heart. In one non-limiting example, the device is configured to be held and positioned on the body by hand. In various embodiments of the invention, two or more sensing components of the device may be connect such that two or more measurements may be obtained effectively simultaneously from two or more skin locations; in one application the first location might be within an area affected by a disease state, such as, for example, skin cancer, while the second location might within nearby normal skin to effectively enable a relative measurement.

In a preferred embodiment, the overall shape of the device is that of wand or pen where the member also provides a means for being held to the skin surface by a clinician performing the assessment. Other means, such as straps, Velcro, belts or a layer of medical adhesive that immobilize the device with respect to the skin surface, are also readily conceivable. Contained within the device, depending on the overall configuration, are necessary power sources, e.g., battery, switches, and electronic circuitry and sensors configured for obtaining capillary and/or cutaneous blood measurements. In certain instances, one or more functions, e.g., data analysis circuitry, power, data display, photonic light sources and sensors, and other components and devices, may be located in a separate portion of the device connected to the member portion by means of electrical wires and/or fiber optics.

Data and analysis from the device may be displayed on a small screen located on the outside of the member in a preferred embodiment. In other embodiments, such data may be transmitted either wirelessly or via electrical connection to adjacent data receiving devices for display, storage and further analysis.

Provided herein, in one aspect, is a method to detect a change in blood microcirculation, the method comprising (a) applying one or more members to the skin; (b) using one or more photonic, localized and discrete excitation sources and one or more discrete or imaging detectors to measure time evolution of one or more blood flow, for example, cutaneous blood flow, parameters in at least one spatially restricted region of the skin; (c) analyzing and quantifying the one or more blood flow parameters; and (d) comparing the one or more blood flow parameters to a data set to determine the absence or presence of a disease state. In some embodiments, two ore more members, containing one or more photonic excitation sources and one or more detectors to measure one or more blood flow parameters are used at adjacent areas of the skin effectively simultaneously, e.g. one containing a suspect lesion of interest and the other a normal (non-disease state) skin. In one embodiment, the data set comprises measured blood flow parameters of at least one skin region, wherein at least one skin region is a reference skin region. A reference skin region includes a skin region having or not having a disease state. In one embodiment, the disease state is cancer. An exemplary cancer is a form of skin cancer. Skin cancer includes stages 0, 1, 2, 3 and 4 of skin cancer. In one embodiment, the method to detect a change in blood microcirculation is performed on both an area of skin of an individual suspected of having a disease, e.g., melanoma, and an area of skin of the same individual which is known to not have the disease, e.g., the reference or control skin region. In another embodiment, a reference region is a region of skin of another individual, wherein the reference region has or does not have a disease state.

Provided herein, in one aspect, is a device for measuring blood microcirculation, the device comprising a sensor comprising at least one photonic localized and discrete excitation source and at least one discrete photonic detector or at least one imaging photonic detector, wherein the sensor is configured to measure one or more blood flow parameters in a spatially resolved manner in a spatially (laterally) defined skin region at various depths. In one embodiment, the photonic detector measures an applied photonic energy absorption by a component of blood. In one embodiment, the photonic detector is an imaging detector. In one embodiment, photonic energy is delivered to and collected from one or more areas of the skin region using optical fibers. In one embodiment, photonic energy is delivered to an area of the skin region from the photonic excitation source. In another embodiment, photonic energy is detected from an area of the skin region with the photonic detector. In one embodiment, the sensor comprises a plurality of photonic detectors, wherein each photonic detector is located at a different distance from the photonic excitation source than another photonic detector.

The photonic detector measures an applied photonic energy absorption by a component of blood. In one embodiment, the photonic detector is an imaging detector. A photonic energy can be delivered to and collected from one or more localized, discrete areas of the skin region using optical fibers.

In another embodiment, the sensor comprises a plurality of localized and discrete photonic detectors, wherein each receiver for a photonic detector is located at different distances from the emission location of the photonic excitation source of the sensor.

In one aspect the area of skin surface illuminated by an excitation light source can be less than 1 mm², between 1 mm² and 5 mm², or more than 5 mm².

In another aspect the area of skin surface measured by the localized discrete photonic detector can be less than 1 mm², between 1 mm² and 5 mm², or more than 5 mm².

In yet another aspect, a device is configured to measure one or more blood flow parameters of an area of the skin region, wherein the area is equivalent to or greater than 0.100 mm in diameter.

A device can be configured to measure one or more blood flow parameters anywhere within of a spatially defined area of the skin region chosen to measure cutaneous blood flow or blood flow parameters, wherein the area is between about 1 mm and about 5 mm in diameter.

A device can also be configured to measure one or more blood flow parameters of an area of the skin region, wherein the area is between about 1 mm and about 30 mm in diameter, 5 mm and about 30 mm in diameter, between about 5 mm and about 25 mm in diameter, between about 5 mm and about 20 mm in diameter, between about 5 mm and about 15 mm in diameter or between about 5 mm and about 10 mm in diameter, between about 10 mm and about 20 mm in diameter, between about 1 mm and about 10 mm in diameter, between about 1 mm and about 20 mm in diameter or between about 10 mm and about 30 mm in diameter.

A photonic excitation source can emit light at wavelengths below 400 nm, between 400 nm and 450 nm, between 450 nm and 500 nm, between 500 nm and 550 nm, between 550 nm and 600 nm, between 600 nm and 650 nm, between 650 nm and 700 nm, or above 700 nm.

In one aspect, the inner member comprises a convex, concave or non-planar surface for to facilitate better contact with the skin. In preferred embodiments of the invention, the depth of skin capillary or vascular measurement provided by such contact is effectively limited to the skin capillary bed by the spacing of discrete sensing elements and measurement area. These dimensions are such that skin blood measurements occur at a depth and region of the skin tissue effectively undergoing blood oxygen exchange with surrounding tissues and not at a depth enabling determination of blood oxygenation parameters reflective of the body as a whole, e.g. arterial blood oxygen levels.

One aspect of this invention is that the area of skin illuminated by excitation light source should be close to the area of the skin from which light is detected thereby enabling to probe preferentially only cutaneous tissue. This feature may be accomplished by spacing of the light source and detector elements on the sensor head surface that contacts the skin surface wherein such elements preferably within 1 mm, more preferably within 0.5 mm and most preferably within 0.2 mm of each other.

Also provided herein is a method to detect a change in blood microcirculation, comprising: providing one or more photonic localized discrete excitation sources and one or more localized discrete photonic detectors to measure temporal evolution of one or more blood flow parameters in a spatially resolved manner within a defined skin region; analyzing and quantifying the one or more measured blood flow parameters from said one or more regions of the skin; assessing said blood flow parameters to identify blood flow; and comparing the blood flow to one or more other assessments to determine the presence of a disease state within said skin region. In some embodiments, the one or more localized discrete photonic excitation sources and one or more detectors are used to measure one or more blood flow parameters.

In one aspect, the disease state is cancer. Cancer, in some instances can be skin cancer that is benign or malignant. In other instances, the cancer is metastatic.

In another aspect, the disease state is hypercholesterolemia, Alzheimer disease, carpal tunnel syndrome, schizophrenia, hypertension, renal disease, type 2 diabetes, peripheral vascular disease, atherosclerotic coronary artery disease, heart failure, systemic sclerosis, obesity, primary aging, sleep apnea, neonatal & adult sepsis, wound healing, or a combination thereof.

In the methods described herein, the one or more other assessments can comprise blood flow parameters measured in a spatially defined skin region or regions. In one embodiment, the skin region comprises a lesion suspicious for cancer. In some instances, the reference skin region does not have cancer.

In such methods, the blood flow parameters are analyzed and quantified.

Analyzing the one or more measured blood flow parameters comprises utilizing spectral analysis such a Fourier or Wavelet transforms, digital filtering of noise, correlation of signals from different skin regions, determination of frequencies and amplitudes and phases of pulsatile signal components.

Assessing blood flow parameters relative to one or more other assessments can comprise comparing signal lifetimes and lifetime distributions obtained from the skin region with a reference skin region.

Analyzing the one or more measured blood flow parameters can comprise determining temporal relationships and correlations between signals acquired from a plurality of photonic detectors or from imaging detector, where each receiver for a localized discrete photonic detector is located at a different distance from the emission of the photonic excitation source.

Analyzing the one or more measured blood flow parameters can comprise determining temporal relationships and correlations between signals acquired from a plurality of photonic detectors at different wavelengths emitted from the localized discrete photonic excitation source.

In such methods, the one or more blood flow parameters can provide an amount of blood flow in the skin region, wherein the amount of blood flow is indicative of the presence of the disease state.

The methods can further comprise performing hemodynamic analyses on a plurality of skin region locations, wherein the hemodynamic analysis of each location is compared to another location to determine or compare disease status.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is illustrative of an embodiment of a cutaneous blood flow monitoring device.

FIG. 2 is illustrative of an embodiment of a cutaneous blood flow monitoring device depicting inner and outer members.

FIGS. 3A-B are illustrative of one configuration of an outer member of a cutaneous blood flow monitoring device.

FIG. 3A is a cross sectional view where the disposable component 304 is configured to seat or guide the attachment of outer member component 301 through its bowl like structure.

FIG. 3B is an end-on view; the structure of disposable component 304 has an opening enabling the inner member 302 to traverse through the outer member components and thereby contact the desired skin region (not shown) in order to enable steady positioning on the skin and blood flow measurements.

FIG. 4 is illustrative of one configuration of a sensing member of a cutaneous blood flow monitoring device.

FIG. 5 exemplifies sensor elements of a sensing member of a cutaneous blood flow monitoring device.

FIGS. 6A-B are illustrative of a sensor of the sensing member of a cutaneous blood flow monitoring device, wherein the sensor comprises a plurality of detectors and one light source.

FIG. 6A is one embodiment of the convex shape of the sensing member structure.

FIG. 6B presents an array of photodetection elements 603 spaced about a single photonic source 602.

FIG. 7 is illustrative of a sensor of the sensing member of a cutaneous blood flow monitoring device, wherein the sensor has an imaging capability.

FIG. 8 is an exemplary illustration of electronic circuitry elements enabling operation of a cutaneous blood flow monitoring device.

FIG. 9 exemplifies representative data obtained using a cutaneous blood flow monitoring device as described herein.

FIG. 9A—normal skin and a confirmed basal cell carcinoma (BCC);

FIG. 9B—normal skin and a confirmed BCC from a different patient

FIG. 9C—normal skin and an irritated seborrheic keratosis.

FIG. 10 shows compares results of spectral analysis of the hemodynamic data obtained from a normal skin and skin cancer (BCC).

### DETAILED DESCRIPTION OF THE INVENTION

Provided herein, in various aspects, are methods and devices for determining cutaneous blood flow within a desired skin region.

Skin microcirculation has been considered an accessible and potentially representative vascular bed to evaluate and understand the mechanisms of microvascular function and dysfunction. Vascular dysfunction (including impaired endothelium-dependent vasodilation) induced by different pathologies is evident in the cutaneous circulation. It has been suggested that the skin microcirculation may mirror generalized systemic vascular dysfunction in magnitude and underlying mechanisms. Furthermore, minimally invasive skin-specific methodologies using laser systems make the cutaneous circulation a useful translational model for investigating mechanisms of skin physiology and skin pathophysiology induced either by skin disease itself or by other diseases such as vascular, rheumatologic, and pneumologic diseases. To date, the skin has been used as a circulation model to investigate vascular mechanisms in a variety of diseased states, including hypercholesterolemia, Alzheimer disease, carpal tunnel syndrome, schizophrenia, hypertension, renal disease, type 2 diabetes, peripheral vascular disease, atherosclerotic coronary artery disease, heart failure, systemic sclerosis, obesity, primary aging, sleep apnea, neonatal & adult sepsis, wound healing, or a combination thereof.

Prior devices described suffer from the absence of (a) adequate reference (control) signal making them sensitive to the type of tissue, physiological state and environmental parameters (e.g., temperature) which introduces significant error due to biological variability and requires complicated calibration and parameterization procedures and (b) lack of spatial resolution that would enable their use within skin lesions. Examples include one such device as described by Howell (U.S. Pat. No. 3,698,382) wherein a platform system provides varying pneumatic pressure to a housing that is placed upon the skin. Within the housing there is optical sensor intended to enable the determination of blood refill rates. As pneumatic pressure is varied, an assessment of capillary refill rate is then made using the optical sensor present within the device; the optical sensor comprises a photonic light source and light detector (also described by Shani et al, U.S. Pat. No. 6,685,635 B2, see below) arranged in such a way that the light source illuminates the skin area surrounding the light detector. Such arrangement of optical sensor enables measurement of bleaching induced by blood displacement, however spatial resolution is lacking because the optical sensor simultaneously collects and integrates light from multiple areas of the skin surrounding the light source; moreover due to its size the devices measures blood flow not only in the cutaneous but also in subcutaneous tissues thereby making assessment on the blood flow within cutaneous lesion only not possible. Alternatively, Shani and Shavit (U.S. Pat. No. 6,685,635) describe a system having an external housing through which pressure is applied resulting in removal of blood from the depressed body region. As pressure is transitorily applied to the external housing, capillary blood refill is assessed using sensors located within the structure of housing. They also report the use of a temperature sensor to improve determination of skin capillary state and overall physiological status. A somewhat different approach is described by Messerges and Hutchinson (U.S. Pat. No. 8,082,017) which combines pulse oximetry with capillary refill time assessment. This device is designed to be placed upon the end of a patient's appendage, e.g., a finger or a toe. When affixed to the patient, one member of the device is located on one side of the appendage and a second member is located on the opposing side of the appendage. Pressure resulting in blood loss is then accomplished by an actuator located in one hinge resulting in both members compressing the intervening tissues; because photonic excitation source and photonic detectors are located on the opposite sides of the appendage, the described device primarily measures blood refill dynamics in subcutaneous tissues and is therefore is not suitable for skin cancer detection.

Other prior devices propose use of multicore optical fibers (Stampoulides et al., US 2013/0123648 A1) for light delivery to the skin tissue for photodynamic therapy and monitoring of treatment. Multicore optical fibers suffer from cross-talk between the fibers making it difficult to use for simultaneous light detection because typically the excitation light source is strong while the light coming back from the tissue is multiple orders lower in intensity; additional complications and crosstalk comes from the need to use specialized interconnects to couple excitation light source and the photonic detectors to the corresponding cores of the multicore fiber. In addition the dimensions and distances between excitation and detection areas at the skin needed for skin cancer detection would necessitate use of very thick multicore optical fibers, making it too rigid and impractical to use in clinical examinations.

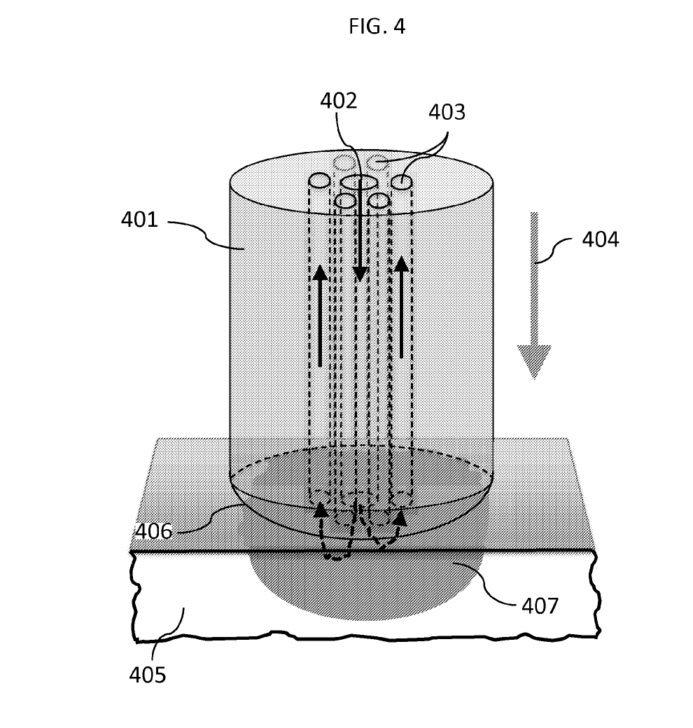

Gaspard et al. describe system for diagnosis of pressure ulcers which include curved sensor tip containing multiple light sources for emitting light towards the skin and multiple light detectors at various distances from the light source for sensing light reflected back from the skin; the sensing tip is made of optically transmissive material (paragraph 35, FIG. 4) which makes possible for light emanating from multiple specific skin areas reach each light detector—therefore such design lacks spatial resolution of the device and method described herein.

None of these devices are specifically constructed as to enable a local determination of skin (capillary) blood perfusion enabling the definition of cancerous from non-cancerous skin tissue. That is, cancerous or precancerous lesions are often of the dimension of a few millimeters or less. All these devices primarily measure subcutaneous blood flow.

The methods and devices disclosed herein overcome the shortcomings of the prior devices which is capable of determining with high spatial resolution the dynamics of cutaneous blood flow, over closely spaced area of skin, e.g., within a mole or suspect cancer growth as compared to an adjacent skin surface. Such devices must be constructed towards this aim and dimensioned accordingly.

Unlike the devices and methods of the previous art, the disclosed methods and devices allow determination of cutaneous blood flow dynamics in the region of the skin located in-between two or more spatially separated areas of the skin defined by the localized and discrete radiation source which irradiates one area of the skin and the photonic detector which monitors radiation emanating from another, closely located area of the skin, both areas being within the skin region of interest.

In some embodiments, variation in amount and rate of blood flow results from a pulsatile action of the heart. In one embodiment, the device member comprises one or more localized discrete sensors and discrete radiation sources. The one or more sensors located, in some instances, within the structure of the device member provide measurements of skin blood flow at one or more instances during recording of blood perfusion. Data from such measurements can be employed for the determination of one or more parameters of blood flow dynamics (generally referred to as hemodynamics). Hemodynamic parameters, in various embodiments, correlate to a disease state. In one embodiment, the disease state relates to the physiology of the individual at the site of measurement, e.g., a skin cancer lesion. In another embodiment, a hemodynamic parameter is reflective of the health of an individual as a whole, e.g., cardiovascular status. In another embodiment, a hemodynamic parameter is reflective of the health of an individual with respect to, for example, hypercholesterolemia, Alzheimer disease, carpal tunnel syndrome, schizophrenia, hypertension, renal disease, type 2 diabetes, peripheral vascular disease, atherosclerotic coronary artery disease, heart failure, systemic sclerosis, obesity, primary aging, sleep apnea, neonatal & adult sepsis, wound healing, or a combination thereof.

### Definitions

A malignant cancer is a cancer that has undergone characteristic anaplasia with loss of differentiation, increased rate of growth, invasion of surrounding tissue, and is capable of metastasis.

Metastatic cancer is a cancer at one or more sites in the body other than the site of origin of the original (primary) cancer from which the metastatic cancer is derived.

A tumor that does not metastasize is referred to as “benign”.

There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma is more likely to invade nearby tissues and spread to other parts of the body.

A melanoma is a malignant tumor of melanocytes which are found predominantly in skin but also in the bowel and the eye (uveal melanoma). It is one of the rarer types of skin cancer but causes the majority of skin cancer related deaths. Malignant melanoma is a serious type of skin cancer caused by uncontrolled growth of pigment cells, called melanocytes. Melanomas also include, but are not limited to, a choroidea melanoma, malignant melanomas, cutaneous melanomas and intraocular melanomas.

Melanoma may be divided into the following types: Lentigo maligna, Lentigo maligna melanoma, superficially spreading melanoma, acral lentiginous melanoma, mucosal melanoma, nodular melanoma, polypoid melanoma, desmoplastic melanoma, amelanotic melanoma, soft-tissue melanoma, and uveal melanoma. Melanoma stages are as follows:

Stage 0—melanoma in situ (Clark Level I).

Stage I/II—invasive melanoma: T1a: less than 1.00 mm primary, without ulceration, Clark Level T1b: less than 1.00 mm primary, with ulceration or Clark Level IV-V; and T2a: 1.00-2.00 mm primary, without ulceration.

Stage II—High Risk Melanoma: T2b: 1.00-2.00 mm primary, with ulceration; T1a: 2.00-4.00 mm primary, without ulceration; T3b: 2.00-4.00 mm primary, with ulceration; T4a: 4.00 mm or greater primary without ulceration; and T4b: 4.00 mm or greater primary with ulceration.

Stage III—Regional Metastasis: N1: single positive lymph node; N2: 2-3 positive lymph nodes or regional skin/in-transit metastasis; and N3: 4 positive lymph nodes or lymph node and regional skin/in transit metastases.

Stage IV—Distant Metastasis: M1a: Distant Skin Metastasis, Normal LDH; M1b: Lung Metastasis, Normal LDH; and M1c: Other Distant Metastasis OR Any Distant Metastasis with Elevated LDH.

In one embodiment, the methods described herein identify a melanoma or a likelihood, or risk of melanoma.

Additional steps or variations in the general method, such as the use of multiple members, different light wavelengths, ultrasound detectors and emitters etc., may be employed within the overall scope of the devices and methods disclosed herein. Accordingly, the scope of the present disclosure is not limited to those series of steps or actions presented and exemplified here.

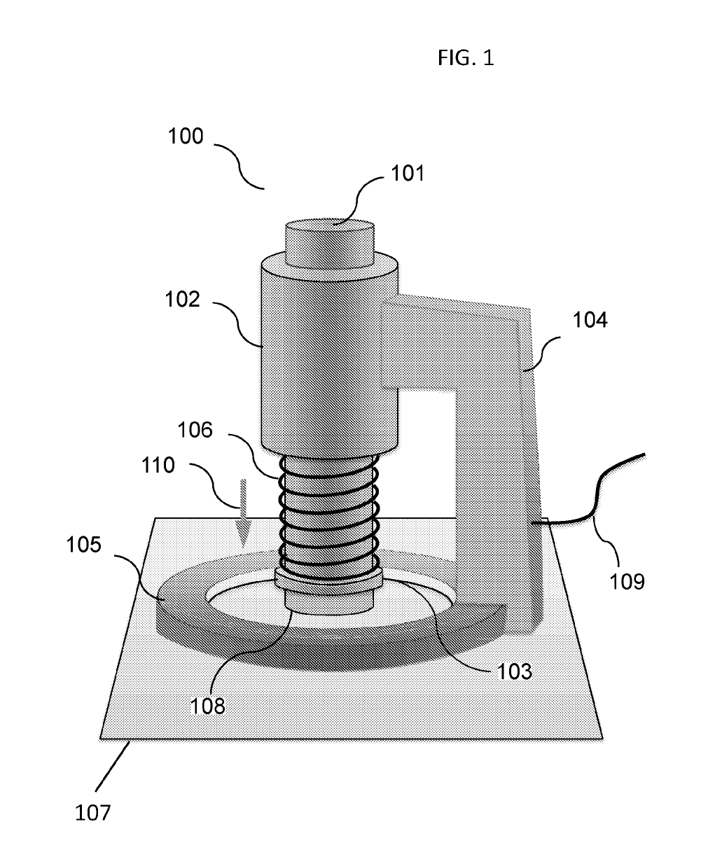

FIG. 1 presents an illustration of an exemplary blood perfusion device, 100. As shown, device 100 has inner member 101 enclosed substantially within a first component 102. The first component 102 is associated with a support component 104 and a base component 105. Collectively, components 102, 104 and 105 comprise the outer member of device 100 and are presented to generally indicate that an outer member of a device may be comprised of multiple components having a variety of functions. For example, the outer membrane component 102 is useful as a guide for inner member 101. As another example, outer membrane component 105 is useful to orient the device for positioning on a specific region of a skin surface 107. As another example, outer member component 104 is useful as a support, enabling the device to house electronics (not shown) and/or photonic sources (not shown) useful for device operation.

Also shown in FIG. 1 is wire 109 extending from outer member component 104. Wire 109 is shown to generally illustrate the functions that may be usefully present in such structures in various embodiments associated by having one or more external connections between device and one or more additional structures, etc. For example, wire 109 may represent an electrical power cord enabling the supply of power to device electrical components. Alternatively, the wire may represent a fiber optic cable transferring photonic energies to and from device 100 to an external unit having photonic energy sources and/or photonic energy receivers with associated electronics enabling signal analysis and processing. A third possibility is that wire 109 represents a data transference means, e.g., USB cable, between device 100 and a separate unit, e.g., a laptop computer or cell phone, enabling data analysis, device operational commands, and display of processed results.

Returning to inner member 101, inner member 101 may be configured to enable measurements of skin blood flow through one or more discrete or localized sensors (not shown) located in inner (sensing) member 101 at component end 108, wherein 108 is a point of contact with a skin surface 107. Also contained within inner member 101 may be additional electronics, etc. to support the measurement of skin properties through one or more sensors located in end 108, and electrical wires, photonic guides and/or other forms of contacts enabling transference of power, data and/or photonic information between inner member 101 and outer member components 102 and 104.

Also shown in this general illustration is a spring 106 and a mounting ring 103 on inner member 101 that are presented to generally indicate the need to provide a means of exerting a small force on the skin surface 107 through the movement of inner member 101 towards and against the skin surface, as indicated by arrow 110. The purpose of this small force is to maintain the contact of the inner ring with the skin through all phases of measurement. Action of the spring 106 located between and in contact with inner member 101 and with mounting ring 103 results in a depressive force on the skin 107 through the pressure of the contact by end 108. The spring constant of spring 106 is chosen to be small enough so that the depression of the skin surface does not result in a forcing of blood from skin capillaries located in the immediate vicinity of this applied force.

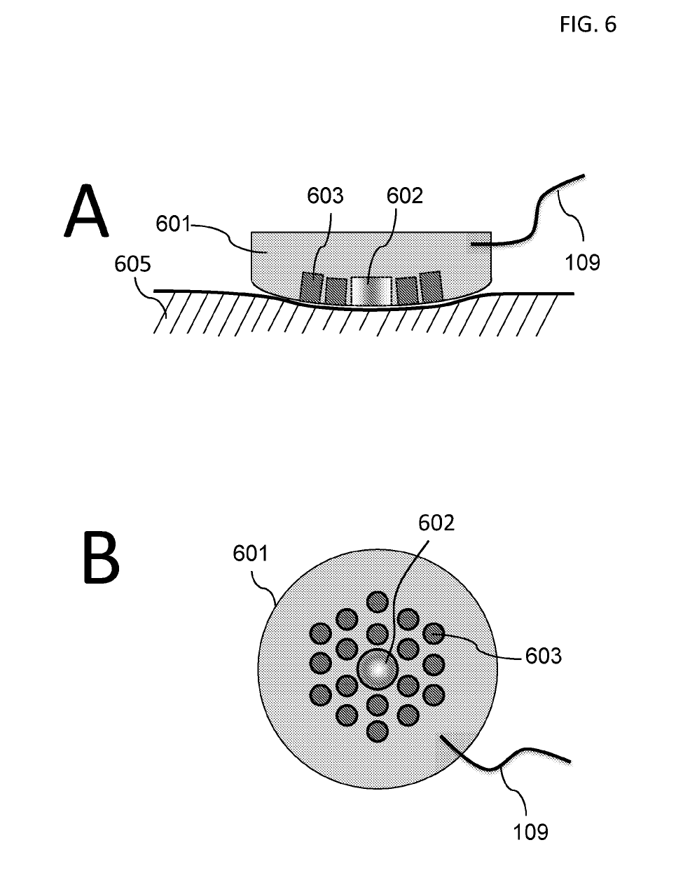

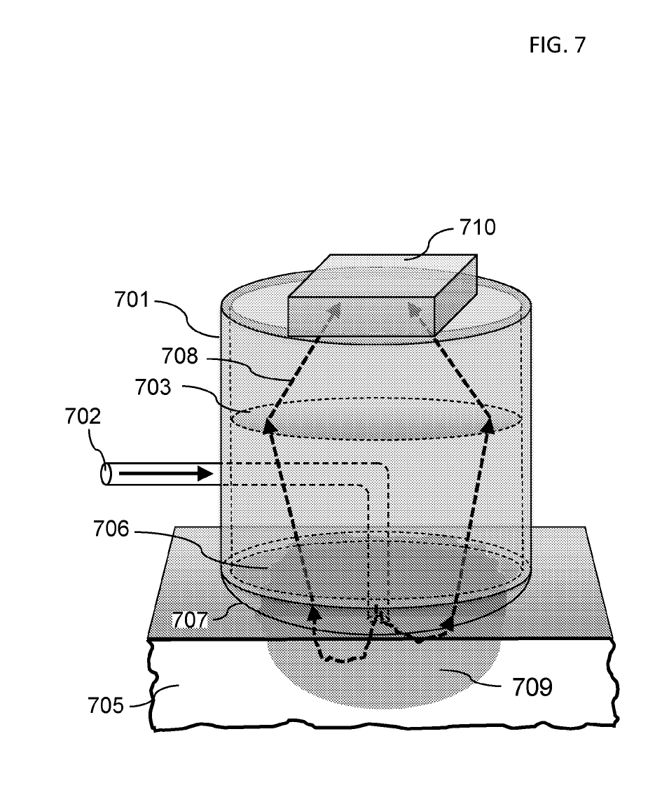

In an exemplary device, the end of the inner member 108 is not subject to motion artifacts. Therefore the end of the inner member 108, containing sensing elements may be permanently attached to the inner member 101 or it may not be attached permanently. For example, 108 could be a component shown in FIG. 7 comprising light sources and detectors. As another example, 108 could be a component shown in FIG. 6; in this case the inner member 101 would apply force to feature 108 and then be retracted from 108, leaving 108 attached to the skin by means of adhesive forces. In the case 108 is not permanently attached to inner member 101, it could be connected by additional cables to enable electronics needed for feature 108 operation.

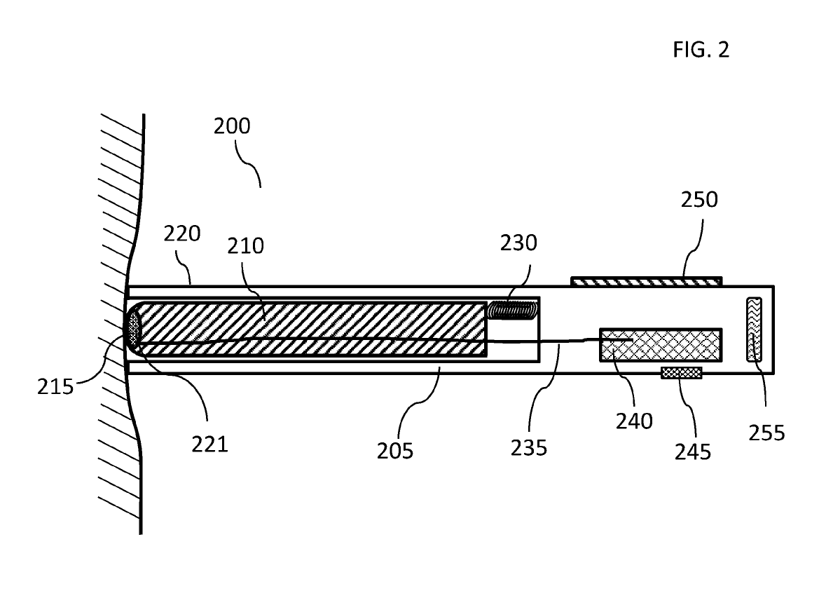

FIG. 2 provides an illustration of an embodiment of a device as provided herein. As shown, device 200 has an outer member 205 that substantially encloses inner member 210, except for an end opening generally indicated by arrow 222. A blood flow sensor head 215 is positioned at the end of inner member 210. Positioned between outer member 205 and inner member 210 is a spring 230, enabling controlled piston-like movement of inner member 210 within outer member 205. The spring 230 is intended to provide a small force to maintain permanent contact of the device with the skin. It should be noted that spring 230 is so configured as to enable retraction of the extended inner member while allowing continuous contact between the skin and the inner member.

In alternate embodiments, the outer member 220 may be affixed to skin, e.g., with use of a medical adhesive, or with belts, Velcro or straps to constrain its position and orientation with respect to skin surface. In certain instances, the structure affixing the outer member to the skin may itself be a portion of the device, e.g., as a separable disposable structure having an adhesive. In other implantation there is no outer member and the inner member with the sensing head is directly held by the hand.

In yet other alternate embodiments, a plurality of inner members 210, sensor heads 215 and/or sensors located within sensor head 215 may be incorporated within device 200 in order to provide a plurality of measurements at one time.

Sensor head 215 at the end of inner member 210 is configured with one or more sensors (not shown) to enable measurement of skin physiological parameters when end opening 222 of device 200 is positioned against the skin. In correct use, device end opening surfaces 220 and 221 are positioned to be substantially in contact with the skin in order to maintain constant contact and pressure to the immediate skin area and measurements of skin blood perfusion to be obtained while doing so. Sensor signals so obtained are conveyed between electronics 240 and sensor head 215 by connector 235.

Also shown within device 200 are operating switch 245, battery 255 and display 250 to enable operation of device 200. Battery 255 may be replaceable, rechargeable, or in certain instances, power to device 200 may be supplied by an external power source, e.g., electrical outlet connected to the device.

Not shown in FIG. 2 are necessary electrical and connections (e.g., optical connections) between the various components of the device 200 to enable their functionality. It will be readily appreciated that such electrical connections as well as electronic circuitry contained within electronics 240 are well understood by those skilled in the art of electronic circuitry.

It may be readily appreciated that the control of mechanical motions and photonic signal delivery and acquisition may be accomplished in a variety of ways and are not constrained to the examples and device component configurations presented here.

Device Operation

In an exemplary mode of operation, a device of the present disclosure, for example, such as one illustrated in FIG. 2, is first positioned on a region of mammalian skin wherein the area to be measured is in contact with end surface 221 of inner member 210. At least a portion of the corresponding end surface 220 of outer member 205 thereby is also caused to come into contact with skin surface within a skin region of interest. In addition, inner member 210 is so constructed to aid in the shielding of photonic sensors contained in sensor head 215 from stray or non-intended energy sources, e.g., stray light.

The operator of the device then activates the device using switch 245. Activation results in electrical power being supplied from battery 255 to electronics 240 and other components, e.g., sensor head 215, display 250, as directed by electronics 240. Upon activation, the data are recorded for a specified time.

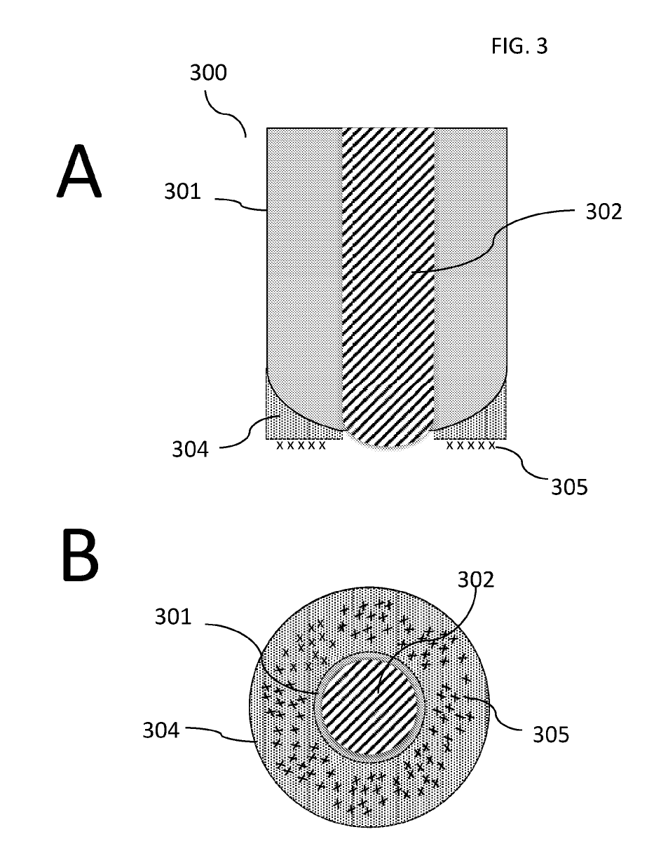

In many implementations, it is a desired feature that the structure or mode of operation of the inner member may be such that access to curved skin regions is facilitated. This may include the shape of the surface that contacts the skin being, e.g., convex rather than planar. Examples of the convex shape of the inner member structure are shown in FIG. 2, FIG. 3, FIG. 4, FIG. 6 and FIG. 7. For example, FIG. 2 shows the rounded end 221 of inner member 210.

In alternate embodiments, the inner member 210 may be contain pressure sensing elements to enable maintenance of constant pressure by an active actuator. Examples of such sensors include pressure transducers so positioned within device 200 or on the device 200 surface as to sense the pressure applied by inner member 210 to skin surface. Such sensors may be present on the inner member 210, outer member 205 or both, the scope is not constrained to any one location. The scope of the present disclosure is not constrained to any one form or method of determining distance traversed.

In addition, pressure sensors located on the outer member in contact with the skin surface may be utilized to ensure that outer member 205 remains in substantial contact with skin surface but is not applying by itself an undesired level of pressure to the local skin area. Readings from such sensors may be sent to display 250 to enable the individual using the device to more appropriately position the device on the skin surface.

In related embodiments, the device may be effectively constructed as a single unit whereby the inner member and outer member form effectively a single contiguous structure. A device having an effectively solid structure, for example the entire structure 200 (FIG. 2) or 601 (FIG. 6) can be securely fixed or held to the skin region for sensing purposes.

In alternate or additional embodiments, a device comprises a plurality of inner members and at least one outer member. In such configurations, a plurality of skin surfaces may be measured in effectively a simultaneous fashion. Such plural forms of the device may be advantageously employed where a suspect lesion is measured during the same measurement period as a non-suspect (control) skin area is measured, without the extended time period required by sequential measurements.

In yet other configurations of the device, the outer member may have at least one element separable from the inner member.

In such embodiments, the outer member may be comprised of one or more separate components, e.g., an adhesive strip having one or more alignment marks to aid in positioning of the inner member and a separate ring or guiding structure to enable the placement of the inner member in a position in accordance with the adhesive strip alignment marks. In yet other embodiments, the outer member may have a conformable portion or separable component, e.g., sponge or soft rubber, element that contacts the skin to promote both good contact of the device with the skin and to provide comfort to the user. Additional forms and types of the structure of the outer members are readily conceivable and therefore the scope of this disclosure is not restricted to the examples and configurations presented herein.

It will be readily appreciated that one or more measurements concerning the presence of blood in the measured region may be made at various points in the measurement cycle. In preferred embodiments, such measurements are made in an effectively continuous fashion, e.g., once every 10 milliseconds or more frequently, such that a contiguous data set describing local blood removal and reperfusion due to action of the heart is obtained enabling detailed characterization of the blood flow dynamics. Data from one or more measurements may then be analyzed to ascertain the likelihood or presence of a disease state.

Exemplary elements of a blood perfusion device are described in greater detail below.

Outer Member

Provided herein, in various aspects, is a cutaneous blood flow device comprising an outer member and an inner member, wherein the device is configured to measure at least one blood flow parameter from a skin region. A primary function of the outer member is to serve as a guide or support to enable the proper positioning and operation of the inner member. As such, in one embodiment, the outer member has at least one surface region in substantial contact with a region of skin proximal to the skin area to be analyzed by the inner member and at least one surface portion able to contact at least a portion of the inner member. In an additional embodiment, the outer member, once positioned against the skin region as a first step in the measurement process, is intended to be relatively stationary during the remaining steps of the measurement process, e.g., remain immobile against the skin, thereby aiding in the guiding of the inner member during the measurement as to reduce motion artifacts. Upon completion of the measurement process, the outer member may be then removed or lifted from the skin surface. This removal may also be coincident with the removal of the inner member, dependent on the exact configuration of the device.

In structure, an exemplary form of the outer member is one that (a) is at least partially conical or cylindrical in overall shape, wherein the inner member is enclosed circumferentially, at least in part, by the outer member and (b) has a surface that may contact the inner member at least at one location via one or more contact points. In many embodiments, such contact points enable the guiding of the inner member to a specific skin location for the application of pressure. In such embodiments, the outer member may have an opening through which the inner member may pass to make contact with the skin region.

In other embodiments, the outer member may have a shape other than cylindrical or conical, e.g., rectangular or C shaped, or even have a shape whereby the inner member is not substantially encircled by the outer member, e.g., the outer member is configured as a linear rail or guide that is configured to serve as a guide to the inner member during inner member operation.

In these and other embodiments, the outer member may be comprised of separable components. For example, at least a portion of one component of the outer member may be a ring or similar conical structure in contact with the inner member. A separate component of the outer member may be in the form of a transparent tape. In this embodiment, the tape may serve as an interface between the skin and the other components of the device, e.g., the other portions of the outer member and the inner member.

In a related or additional embodiment, the outer member has a separable component that has both a guiding function as well as an adhesive function. FIG. 3 presents an example of one such embodiment. FIG. 3 presents a section of a device 300 having an outer member component 301 in contact with inner member 302 and disposable outer member component 304. Disposable component 304 has adhesive 305 to facilitate the positioning and adhesion of device 300 to the skin in a desired location. As shown in FIG. 3A, a cross sectional view, the disposable component 304 is configured to seat or guide the attachment of outer member component 301 through its bowl like structure. As shown in FIG. 3B, an end-on view, the structure of disposable component 304 has an opening enabling the inner member 302 to traverse through the outer member components and thereby contact the desired skin region (not shown) in order to maintain constant contact with the skin during measurements.

In such instances as those illustrated in FIG. 3, a separable component may be constructed as a disposable component such that it may be employed on a single use basis. A desirable feature of such embodiments is that the disposable component may be positioned onto the skin in advance of the attachment to this disposable component by the other portions of the outer member by the operator.

One requirement for such separable components, e.g., a tape, collar or other disposable component, is that it be so constructed as to enable the operation of the inner member, e.g. the measurement of blood dynamics within the skin by one or more sensors located within the inner member. In those instances wherein a separable component, such as a tape, intervenes between the skin surface and a device component such as an inner member having motion and/or sensing capabilities, in many embodiments, it is desired that the separable component be relatively thin (e.g., less than 0.2 mm in thickness) and conformable or stretchable to the movements and applied pressures by the device component (e.g., inner member) as well as able to pass signals employed in measurement (e.g., the separable component is effectively transparent to the wavelengths of light utilized for photonic measurements). In one embodiment, separable component 304 is made of a flexible material that can easily compress to conform to a convex probe head shape of a device component (e.g., outer member).

In certain instances, an outer member is constructed to be affixed to the skin and then disposed of after use. In one embodiment, this disposable outer member may also contain one or more blood sensors, e.g., photonic sources and/or photonic receivers. For embodiments such as these, sensors may be fabricated or positioned within a tape or other separable component using one or more methods of construction, such as printed electronics, whereby the circuitry elements and sensors are effectively printed into the structure of the separable component, e.g., the tape.

The outer member, in various embodiments, is configured to enable the positioning of the device by hand on the intended region of the skin of an individual for blood flow measurement. Accordingly, all or a portion of the outer member may be constructed in the form of a handle or similar structure enabling its manual placement and operation. For example, a device with the outer member in the shape of a pen or rod-like structure generally sized between about 7 centimeters and about 15 centimeters in length and from about 1 centimeter to about 4 centimeters in approximate diameter would enable clasping of the outer member of the device by hand for use in positioning and device operation. It would be understood that alternate sizes and holding arrangements are conceivable and the dimensions of the device are not restricted to those described here.

Alternatively or additionally, the outer member may be constructed with differing functional sections. That is, a portion of the outer member may be configured to be held by a hand, while a separate portion of the outer member is configured to interact (e.g., as a guide and/or as an anchoring point for forces to be applied) with the inner member.

In still other embodiments, the outer and inner members are immobilized relative to each other such that force applied to the outer member results in pressure being applied to the skin by the inner member. In such instances, the outer member may be structurally indistinct from the inner member, i.e., the inner member is distinguished by the presence of one or more sensors and the outer member has one or more circuitry elements needed for data measurement and display, wherein both the inner and outer members are housed within the same overall shell or covering.

In various embodiments, the outer member and/or inner member may also contain components or structures enabling the transference of data, processed data, power and/or sensor (photonic) energy to and/or from a unit separate from the device. For example, the outer member or inner member may be configured for attachment to a USB cable enabling transference of measured data with a separate unit, e.g., a cell phone, for control/operation instructions, signals, additional data processing and display of results. In an alternate example, the outer unit may be configured for attachment to a fiber optic cable, enabling the transference of photonic energy to and from the outer member and then to the inner member and/or sensors.

In additional or other embodiments, the outer member or inner member may comprise one or more controls and/or display or alerts. Examples of these may include one or more on/off switches or buttons for initiating and/or operating the device, one or more indicator lights indicating the operational status of the device, one or more audible alerts indicating the status of the device or instructional activities to be performed, one or more small displays configured to display operational status, data and/or the results of data process, and any combination thereof.

The outer member may be constructed of a variety of materials, e.g., plastics, rubbers, metals. The outer member may have electronic circuitry, batteries, lights, displays, etc. The exact composition of outer member materials is dependent on the nature of the device embodiment and functional needs. Creating such constructions are well known to those skilled in the art of medical device construction.

Inner Member

Provided herein, in various aspects, is a cutaneous blood flow device comprising an inner member and an outer member, wherein the device is configured to measure at least one blood flow parameter from a skin region. In various embodiments, a primary function of the inner member is to position the sensing elements onto a desired skin or tissue region. The outer member may be optional.

Overall, the region of skin to be measured is desired to be of a dimension suitable for the measurement of cutaneous blood flow (e.g., skin capillary blood flow) during a process described herein by the sensor methodology employed. Accordingly, the dimensions of the skin contact region (and corresponding inner member surface) are, in various embodiments, preferably greater than that represented by a single capillary, i.e. 6-8 microns in cross section. In many embodiments, a desired function of the device, in part, is the ability to distinguish between normal capillary networks and those associated with cancerous tissues, wherein the dimensions of the contact region are therefore more preferably greater than that of a single capillary. Accordingly, in many embodiments, the dimensions of the skin contact region and of the corresponding surface of the inner member are at least 0.1 mm² in area.

In exemplary embodiments, the shape of the inner member that contacts the skin region (or contacts a portion of an outer member, e.g., a tape, intervening between the inner member and the skin region) is configured to facilitate the maintenance of steady contact with the skin region.

In an additional embodiment, the inner member serves as a support or structure housing one or more sensors which enable the determination of blood flow in a measured region of skin. In an exemplary embodiment, such sensors comprise at least one source of photonic or ultrasonic energy, to be applied to the skin region, and at least one means, e.g., waveguide, fiber optics, etc., of receiving one or more photonic energies from the skin region. A non-limiting example of such an arrangement is shown in FIG. 3.

FIG. 3 illustrates a section of an exemplary inner member 301, having rounded tip 306. Within inner member 301 is fiber optic 302 providing photonic energies to a skin tissue region 307 located within larger skin tissue region 305. Photonic energies so delivered may scatter and be absorbed in region 307. A portion of these energies may in turn encounter return fiber optics 303, conveying these photonic signals to one or more photodetectors located elsewhere in the device, as shown by arrows directed upward. Blood flow parameters are measured from tissue region 307 during the conditions associated with blood hemodynamics due to the pulsatile action of the heart modulating the amount of blood in this region.

One may readily envisage embodiments where a plurality of photonic energy sources and/or photonic energy signal receivers (photodetectors) are utilized to better inspect larger skin regions and/or comparatively assess under the same measurement cycle various discrete skin areas within a larger site of measurement. For example, if a desired function of the device is to delineate the margins of a tumor, then by use of an array of sources and detectors, e.g., employing multiple fiber optic cables with multiple sources and photodetectors, one might effectively image the boundary or signals associated with the transition of capillary types associated with cancerous tissue versus normal tissue.

In order to accomplish the desired functions of the inner member, the inner member may be composed of a variety of materials and components. For example, materials such as plastics, rubbers, metals such as stainless steel, aluminum, brass, may be employed in various combinations in order to configure the inner member according to the requirements of that embodiment of the device.

In alternate or additional embodiments of the device, the inner member can be used alone without the outer member. In such a case the inner member would preferably be a short, round or square part, for example as the part 601 depicted in FIG. 6. In one embodiment, the surface of 601 in contact with the skin comprises an adhesive coating or an adhesive consumable part which would maintain the surface of 601 in the contact with the skin throughout a measurement of a blood flow parameter.

Sensors

Provided herein, in various aspects, is a blood perfusion device configured to measure at least one blood flow parameter from a skin region using one or more sensors. In various embodiments, the sensor comprises a discrete and localized energy source or transmitter, such as a photonic excitation source. The active area of the localized discrete energy source or transmitter can be less than 1 mm², between 1 mm² and 5 mm², or more than 5 mm². In various embodiments, the sensor comprises an energy receiver or detector, such as a photonic energy signal receiver or photodetector. A photonic energy detector can be a discrete, localized detector or it can be imaging detector. The active area of the localized discrete photonic detector can be less than 1 mm², between 1 mm² and 5 mm², or more than 5 mm². In some embodiments, the sensor is a component of an inner member of the device. In other or additional embodiments, the sensor is a component of an outer member of the device. In some embodiments, the device comprises a plurality of energy sources. In other or additional embodiments, the device comprises a plurality of energy detectors or a imaging detector. A principal element of the device of the present methods and devices disclosed herein is the incorporation of at least one localized sensor intended for the measurement of a blood flow parameter, including, but not limited to, blood volume and perfusion rates in the spatially defined measured skin region, e.g., the skin blood vessels. In general, such sensors may utilize the transference of one or more energies to and from the body region where such energies are chosen based upon their interaction with one or more aspects of biological tissues appropriate for the determination of skin capillary blood perfusion.

Generally, such energies are preferably supplied to the immediate body region by a transmitter located in or on the device. Following interaction with one or more body tissues, structures and/or chemical components, a portion of the non-absorbed energy may then be radiated back from the body region to be received by a receiver on the device. The resultant data may then be analyzed for signals associated with one or more components of blood, e.g., hemoglobin, or blood vessels, associated with capillary blood perfusion.

In preferred forms of the methods and devices disclosed herein, such energies are photonic in nature, e.g., signals at one or more visible wavelengths that are absorbed, in part, by chromophores contained within the hemoglobin of blood. In order to supply such photonic energies, a source such as a light emitting diode is typically employed. Such sources advantageously provide light centered about a single frequency, e.g., 590 nm±20 nm or 420 nm±20 nm, which may be selected for its sensitivity to one or more blood components and/or insensitivity to other biological structures or chemical compounds within the skin or to enable measurement at various depths in the tissue (see below).

Such ranges of light may be obtained by use of one or more filters within the light path from the light source to the detector, e.g., as a band pass filter position in front of the photodetector, or photonic detector, element of the device. Such positioning may also advantageously limit the introduction of unwanted light to the photodetector where such light arises from a source other than that of the device, e.g., light at other wavelengths arising from light sources present elsewhere, such as a room light. In addition, the use of filters assists in assuring that intended photonic energies are measured, e.g., filters that only enable polarized light to pass may be employed within various embodiments of the device.

It should be understood that a variety of chromophores are present within biological tissues including blood and accordingly, the scope of the present methods and devices disclosed herein is not restricted to any one wavelength or wavelengths for the determination of blood presence within the measured region. Likewise, a plurality of photonic energies, i.e., different wavelengths, may be employed to enable more detailed analysis of the capillary blood perfusion. In certain instances, such different wavelengths may be selected to enable various depths of measurement, i.e., certain frequencies penetrating to deeper tissue regions than others, to enable a three dimensional interpretation of capillary perfusion and density.

In alternate embodiments, sensing at different wavelengths can be used to implement ratiometric detection to facilitate separation of contributions from light scattering by the tissue and absorption by the blood and to suppress motion artifacts. Sensing employing different wavelengths may utilize common or differing structures for the delivery of photonic signals to the skin surface, e.g. multiple LEDs utilizing a common fiber optic for delivery of differing photonic signals of differing wavelengths. In addition, the various light sources may be rapidly turned on and off such that signals from one wavelength do not interfere with those of another during the course of a measurement cycle.

In addition, photonic energies responsive to blood components other than those in the visible wavelengths may also be employed. Such energies may include near infrared, mid-range infrared or ultraviolet.

In certain instances, photonic energies may interact with tissue structures or components not directly present within the blood, e.g., melanins within the skin cells themselves that may provide indirect indices of blood volume/vessel arrangement and/or tissue structure associated with a disease state.

To provide the necessary photonic energy, alternate means other than light emitting diodes are readily conceivable. Such alternate sources may include vertical cavity semiconductor lasers, liquid crystals, incandescent bulbs, organic light emitting diodes or halogen lamps. Accordingly, the present invention is not restricted to any one form or type of photonic source. In alternate embodiments, an ambient light may be used with a desired detection wavelength selected using appropriate optical filter in front of the photonic detector.

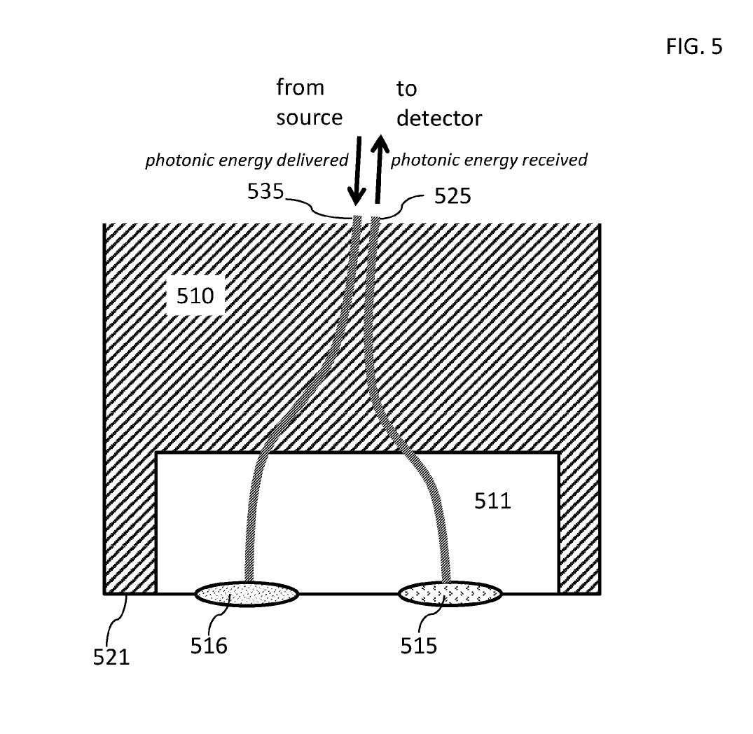

In exemplary embodiments, photonic energy is delivered to a sensor head located at the end region of an inner member by one or more photonic sources. An example of such an embodiment is shown in FIG. 5, which presents sensor elements of a device provided herein. As shown, the end of inner member 510 contains sensor head 511, which is a structure employed to mount one or more sensing elements within the inner member. Photonic energy is conveyed to lens 516 by fiber optic cable 535. Photonic energy may then be received through lens 515 and conveyed back to a photodetector or other forms of light sensitive structures located elsewhere through fiber optic cable 525. The structure of inner member 510 is enclosed, in part, by outer member 505. Surfaces generally indicated by surfaces 520 and 521 are regions intended to contact skin surface at least in part during operation of the device. It should be understood that within the scope of the present methods and devices disclosed herein, sensor head 511 may not be configured as a separable element distinct from inner member 510.

In this embodiment, lens 516 and 515 may serve to collect and orient photonic energies between the device and the skin surface. Such lenses may be constructed as separate components or be constructed from a larger structure, e.g., by polishing the end of an optic fiber used to transfer photonic energy. In addition, the lens (or other photonic lens or guide) may be configured in or angled in relationship to the surface to optimize signal transmission into body tissue.

Another example may be shown by FIG. 2, wherein photonic energy is delivered to a sensor head 215 by one or more sources located within the electronic component 240 whereby the photonic energy is transmitted to the sensor head via one or more fiber optic cables, represented by connector 235. In other embodiments, the photonic source is located within sensor head 215 and connector 235 serves to supply electrical signals governing the activation of the photonic source. It should be understood that within the scope of the present methods and devices disclosed herein, sensor head 215 may not be configured as a separable element distinct from inner member 210, e.g., a single structure may comprise both functionalities.

In this embodiment, one or more lenses may be employed to collect and orient the emission of photonic energy from the device into the energies between the device and the skin surface. Such lenses may be constructed as separate components or be constructed from a means for conveying the photonic energy to the surface of the sensor head, e.g., by polishing the end a fiber optic. In addition, the angle of the lens, fiber optic (or other photonic lens or guide) may be configured in, or angled in, relationship to the surface 221 to optimize signal transmission into body tissue.

To receive photonic energies after being transmitted into the body tissue, one or more detectors responsive to photonic energy are employed in preferred embodiments of the present methods and devices disclosed herein. Such detectors typically are comprised of one or more semiconductor devices, e.g., photodiodes, wherein the photonic energy is converted to an electrical signal. Other forms of detectors are conceivable, e.g., photomultipliers, and the scope of the methods and devices disclosed herein are not constrained to any one type of photonic energy detector.

In preferred embodiments, one or more photodetectors are located within device electronic circuitry. In such instances, the received photonic energies are transmitted through a fiber optic cable or a fiber optic bundle and connector to the appropriate electronic circuitry and components. Accordingly, in some embodiments, the device may be comprised of a plurality of optical fibers to enable both emission and reception of photonic energy, with various fibers constrained to either emission or reception.

In alternative embodiments, one or more photodetectors may be positioned within the sensor head. In such instances, a connector may serve to convey an electrical signal from the sensor head to electronic circuitry.

As with the emission of photonic energy, in one embodiment, one or more lenses may be employed within the sensor head to orient the received photonic signal to enable subsequent detection and signal analysis. Such lenses may be separate components or a portion of a component, e.g., a polished end of an optical fiber. In addition, the angle of the fiber optic (or other photonic lens or guide) may be configured in relationship to the surface to optimize signal reception from the body tissue.

In preferred embodiments, the sensor head photonic emission source at the surface of the sensor head is positioned in general proximity to where a receiver of the photonic signal is located. In preferred embodiments, an emitter/receiver pair is located in close proximity to each other and effectively flush with the surface of a sensor head, as shown in FIG. 5. The spacing between emitter and receiver is preferably such that the photonic signal propagates in large part through the adjacent skin and tissue and, in one embodiment, is confined primarily to the skin.

In preferred embodiments, the solid elements comprising the components where light is emitted from the sensor head and resultant signals are received, e.g., the lens, are effectively flush with the surface of the sensor head such that an effectively planar surface over the entire surface is achieved.

In alternate embodiments, one or both of the components may be slightly recessed or slightly extruded relative to the surface sensor head. Such arrangements may lessen the likelihood of immediate transfer of photonic signal from emitter to receiver thereby reducing available signal from the body or enhance blood displacement upon application of pressure.

In other alternate embodiments, both signal emitters and receivers are located at the distance from the skin surface providing means to perform measurements of photonic energy reflected from skin; furthermore in the reflectance configuration the use of photonic energy at different wavelengths is desired, thereby providing means for ratiometric determination of time-dependent changes in an effective skin color in response to mechanical or temperature-induced perturbation of skin surface. Skin color changes are one of the indices sensitive to changes in skin capillary density and blood flow.

In general an opaque or material that does not result in significant transference of photonic energies or fluorescence in response to photonic energies employed is desired to comprise the structural aspects of the sensor head, i.e., device components not including those through which photonic signals are intended to travel; this is needed to enable spatial resolution of the measurement so that photonic detectors detect light from only a small area of the skin. Likewise, other aspects of the device, e.g., sections of the outer member and sections of the inner member are generally preferred not to be constructed of materials that transmit photonic energies in the utilized frequencies nor fluoresce in the utilized frequencies, if these sections of these components may be within the photonic path or otherwise interact with the photonic signals.

In other embodiments, the photonic source(s) and photodetector(s) may be located at or in near proximity to a device surface that is intended to contact a skin region for measurement. An illustrative example of one such embodiment is shown in FIG. 6. Panel A of FIG. 6 presents a cross section image of a plurality of photodetectors 603 in near proximity to a photonic source 602. Note that photodetectors 603 and photonic source 602 are at the surface of an inner member 601 and are intended to interact with skin and tissue 605. Not shown are electrical elements, e.g., wires, providing power and signal data between the photodetectors and photonic source and controlling circuitry located elsewhere.

Arrangements of photonic signal emitters and receivers may include other forms than pairs, e.g., a plurality of receivers to a single emitter or the converse. In other instances, varying numbers and arrangements of receivers and emitters may be employed in a pairwise or non-pairwise fashion or organization. Such arrangements may serve to increase the sensitivity of the device and thereby enable a reduced power of photonic energy to be employed, which in turn may further restrict the measured region to the skin vasculature rather than deeper tissues. FIG. 6B illustrates this point by presenting an array of photodetection elements 603 spaced about a single photonic source 602. Alternatively, arrays of emitters and receivers (or sources and detectors) may be employed. These arrays may serve to provide a two dimensional map of a skin region blood flow. In such instances, various spatial combinations of emitters and receivers may be employed sequentially to provide insight into overall blood vessel arrangement, density and depth and enable simultaneous measurement-based comparison of a lesion area and a healthy surrounding tissue. In other embodiments, various types and spacing of emitters and receivers may be employed to facilitate the use of one or more wavelengths of photonic energies, effectively simultaneously, for enabling the examination of overall blood vessel arrangement, density and depth.

In yet another embodiment, multi-element photodetectors such as those employed in electronic cameras, e.g., charge coupled devices (CCDs), may be employed as a component of a photonic energy sensor. In such instances, a larger area may be simultaneously measured without the need for multiple fiber optic lines or multiple photodiodes. An example of this form of embodiment is presented in FIG. 7. FIG. 7 presents the portion of an inner member 701 that contacts skin and tissue 705 at inner member surface 707. In this instance, photonic energy is supplied to the skin and tissue region 709 via optic fiber 702 in the direction of the solid arrow. Upon scattering in the tissue of the body region 709, the transmitted light indicated by dashed arrows 708, is collected through the surface of inner member 707 and relayed through lens 703 onto CCD 710. In this example, the composition of structure 706 comprising at least a portion of inner member 701 is effectively transparent, allowing photonic energy 708 to transit from skin region 709 to CCD 710.

The scope of the present methods and devices disclosed herein is not restricted to the use of photonic energies for the determination of amount of blood, blood capillary density, perfusion rate or volume, and blood flow dynamics, either directly or indirectly, in the measured region. Examples of other such energies include, but are not limited to: electromagnetic (radio wave) energy in gigahertz or terahertz frequencies, or high frequency ultrasonic energies.

A sensor sensing the relative displacement of the inner member relative to the outer member may be employed to aid in the measurement process to enable reduction of motion artifacts. That is, the distance traversed by the inner member relative to the outer member once the outer member is positioned against the skin may serve to aid in the operation of the device.

Signals associated with temperature may serve as additional metrics regarding the physiological status of the measured region. For example, it is well known that blood flow to the skin surface may be significantly lessened by cold temperatures due to vasoconstriction. Conversely, blood flow to the skin may be significantly enhanced in those scenarios where the body or regions of the body are attempting to shed heat, i.e. skin vasodilation. In such instances, the use of a temperature sensor, e.g., a thermocouple, positioned on the sensor head to contact the skin may provide data useful in the analysis by enabling corrective terms to be employed. A second temperature sensor positioned elsewhere may also be employed to provide additional useful temperature data, e.g., ambient air temperature, which may be employed in the subsequent data analysis.

In a somewhat different use of temperature, the area to be measured may be intentionally chilled and the recovery of blood perfusion to the region monitored with a device of the present methods and devices disclosed herein. In such instances, the body's vasoconstriction actions serve to limit blood flow to the immediate region. Accordingly, in such embodiments, a device of the present methods and devices disclosed herein may simply monitor the immediate region as the region warms up and blood re-perfuses the region without movement of the inner member. Alternatively, in such embodiments, the inner member and outer member may be constructed as a single unified structure, one in which the inner member is incapable of differential movement with respect to outer member. To chill the skin region, a component such as a Peltier thermoelectric cooler may be incorporated into the device, in particular into one or more areas of the device intended to contact the skin. Alternatively, external cooling means, e.g., an ice cube held against the skin, may be employed.

Similarly, heating the measurement region can also provide additional information disease status in the region of interest. Various means, e.g., heating elements, may be employed to heat the skin region. Similarly, cycling the temperature in conjunction with the measurement can also provide additional information about disease status in the region of interest.

Additional sensors may be included in the device or components of the device, e.g., adhesive tape having one or more sensors incorporated or added, to aid in the operation of the device and/or determination of skin region physiological status. These sensors include but are not limited to: biochemical sensors, e.g., for secreted biomolecules indicative of a disease state, pressure sensors, temperature sensors, pH or ionic sensors, electrical e.g., capacitive sensors, and position or motion sensors, e.g., that aid in a more effective mapping of the boundary of a suspected skin lesion.

In yet other embodiments, video and/or audio sensors may be employed to facilitate device placement and correct alignment on a skin region, e.g., a suspected lesion, or to provide additional information regarding blood and/or disease status. For example, use of a video sensor, e.g., a small camera attachment, may help assist the orientation of the device on the patient or enable the automatic recording of the lesion image in one or more wavelengths of light. Such images, e.g., the overall color or heterogeneity of appearance may assist in the diagnoses of a disease state. Likewise the use of one or more highly sensitive audio pickups or microphones located on or near the device surface in contact with the body may enable additional information regarding blood flow in the general area being measured.

In various embodiments of the present methods and devices disclosed herein, one or more sensors and sensor types may be employed within a device to provide data, enabling the assessment of blood within the measured region.

A number of sensors are conceivable and accordingly, the nature and type of sensors that may be employed within the scope of this disclosure are not limited to those examples and embodiments presented here.

Electronics

In various aspects, in order to enable the functions of the devices provided herein, one or more electronic components are utilized. In exemplary embodiments, a device comprises an inner member and an outer member, wherein the device is configured to measure one or more signals indicative of blood perfusion. In general, these electrical components may govern the automated movement of the inner member relative to the outer member or skin surface, the activation of one or more sensors useful for the determination of blood flow at one or more time points, the analysis and display of results, and any combination thereof.

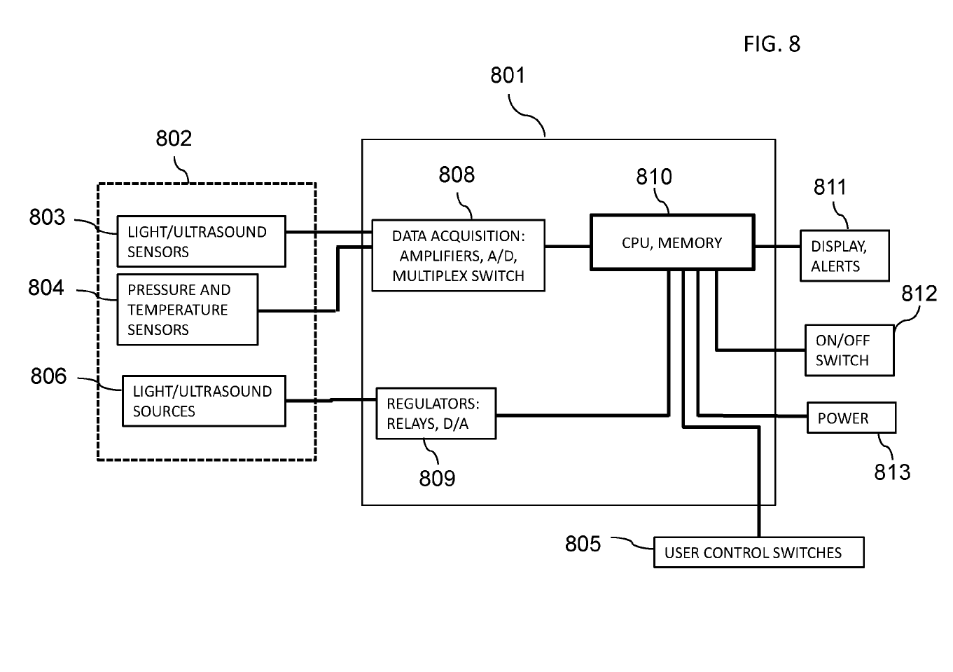

A representative illustration of electronic circuitry elements enabling such functionalities is presented in FIG. 8. As shown in FIG. 8, contained within electronic circuit 801 is a processing unit 810 having memory. Also present in circuit 801 are other components, e.g., components for digital signal acquisition 808 such as multiplex switch, analog to digital converter, and amplifiers; and components to generate signals 809, e.g., power regulators, relays and digital to analog converters. Such components are typically employed for supplying regulated power to one or more sensors, receiving data from such sensors, as well as the control of electrically operated elements.

Sensors and electrical elements that may be employed by circuitry 801 are generally indicated by the group delineated by box 802 and include, without limitation, photonic and/or ultrasound sensors 803, pressure and temperature sensors 804, photonic and/or ultrasound sources 806.

In addition to sensors and electrical elements, circuitry 801 may also have additional inputs and outputs, including, but not limited to, power input 813 (e.g., battery), on/off switch 812 governing overall operation of the device, user controls 805 enabling staged operation of the device, and display or alert 811 for conveying device status and/or measurement data and results via visual and/or audible means.

It will be readily understood that the exact nature and arrangement of circuitry elements will be particular to that embodiment of the device and the example presented here is solely to illustrate the forms and types of circuitry elements enabling the control and operation of a typical device embodiment. Additional components, sensors, actuators, etc. may all involve various permutations of the components and elements presented here and accordingly the scope of the present disclosure is not restricted to that presented in this example.

Likewise, one skilled in the art of electronics will readily appreciate that various elements of the electronic circuitry can be located in various components of the device in order to better enable the overall functionality of the device. For example, certain electronic circuitry elements associated with the control of device operations may be located in the outer member whereas initial signal processing may be located in the inner member. In yet other embodiments, a portion of the data analysis and display may be located in a unit in wired or wireless contact with either the inner or outer member. The exact nature of the placement of electronic elements is therefore governed by the form and requirements of the device embodiment and therefore, the scope of the disclosure is not restricted to any one method or structure for the arrangement of electronic elements.

Data Analysis

In various aspects, data obtained by a device of the present disclosure enables a description of one or more parameters associated with blood flow and/or quantity in the spatially defined measured region (e.g., skin region). Exemplary parameters include, without limitation, the dimensionality of vasculature, vascularization density, flow resistance, ability of cutaneous blood vessels to vasodilate or vasoconstrict in the measured region, spatial heterogeneity of blood flow within the measured skin region and any combination thereof. Such parameters are useful in the determination of a disease state such as skin cancer when the parameters are compared to normal, non-malignant skin. Furthermore, in various embodiments, it is a desired feature of the present methods and devices disclosed herein that the data obtained by a device of the present disclosure enables a description of the dimensionality and/or quantity of capillary blood vessels in the measured skin region through the measurement of blood capillary perfusion and other related parameters. Such indices are useful in the determination of a disease state, such as cancerous or precancerous states, e.g., a melanoma, as compared to normal, non-malignant skin. That is, it is well known that skin cancers often have a denser capillary network or larger dimensioned vasculature relative to those present in non-cancerous skin or common nevi. For example it has been shown that mean vascular counts in cutaneous malignant melanoma are up to ˜324% higher than in common acquired nevi. Moreover a gradual rise in vascularity with tumor progression was observed offering a basis for early detection and for monitoring efficacy of treatment.

Accordingly, assessment of the rate and amplitude by which blood perfuses a skin region may serve as useful tool in discriminating between a cancerous, atypical and non-cancerous state.

Induction of angiogenesis generally provides a supply of nutrients and oxygen for malignant tissue growth, invasion, and metastasis. In order for a tumor cell to survive, it cannot be more than a few hundred micrometers from the nearest blood vessel. Blood vessel structural abnormalities have been shown to reveal underlying disease very early during the onset of disease; for example, after arrival of only 60 to 80 of tumor cells to an in vivo host tissue it starts to exhibit atypical changes in vasculature and that these changes extend beyond tumor margins. Skin cancers have a denser capillary network or larger dimensioned vasculature relative to those present in non-cancerous skin or common nevi.

For example, mean vascular counts (MVC) in cutaneous malignant melanoma are up to ˜324% higher than in common acquired nevi and ˜500% higher than in normal skin. Similar increases in MVC may occur in BCC and SCC tumors. Moreover a gradual rise in vascularity with tumor progression offers a basis for early detection, for monitoring efficacy of treatment and prognostic value. Neovascularization in melanoma correlates with poor prognosis, mortality, and elevated rate of relapse. Measurements of passive blood perfusion using high-resolution laser Doppler perfusion imaging exhibit significantly elevated blood flow in primary melanoma tumors as compared to dysplastic melanocytic nevi (2.2×) and normal skin (3.6×); increase blood flow occurs in BCC tumors. Thus, one useful parameter that may be determined within the scope of the present disclosure is a relatively higher amount of blood being present in a suspect region, e.g., the tumor as compared to adjacent normal skin. Therefore, blood volume represents a target parameter that can potentially be used for diagnostic purpose at one or multiple time-points.

Blood flow rate in a tumor is proportional to (a) pressure difference between arterial and venous side and (b) inversely proportional to viscous and geometric resistance of a vascular network. Pressures on the arterial side of tumor and normal tissue are equal, however pressures in more dominant venular vessels of the tumor are significantly lower than in the normal tissue. Moreover many sold tumors have highly elevated interstitial fluid pressure (IFP) which is attributed to leaky capillaries, increased resistance to interstitial fluid flow, and impaired lymphatic drainage. IFP in combination with lower venular pressure has been implicated in being responsible for the vessel collapse, the flow stasis and reversal in tumor vasculature.

Vascular resistance to blood flow in cutaneous cancers is higher by one to two orders of magnitude than in surrounding normal tissues; this increase is due to various factors such as changes in diameter of blood vessels, disorder in the geometry of the vascular network and increased tortuosity of the vessels. Tumor tissues are known to develop vascular networks with major geometrical abnormalities such as heterogeneous vessel distribution, a lack of vessel hierarchy, increased intervessel distances, arterial to venous shunts, excessive branching, and blind vascular ends. Geometrical flow resistance of tumors is nonlinear function to applied pressure. The flow resistance is significantly higher at lower perfusion pressure and then asymptotically decreases to a constant value at higher pressures; such non-linear flow dependence in tumors is in contrast to a constant flow resistance of normal tissues and has been attributed to viscoelasticity of tumor vessels and to cellular pressure exerted by the surrounding tumor cells. Thus, relative changes in a blood flow rate and overall temporal dynamics of blood flow through tumor vasculature as compared to normal vasculature may represent second set of kinetic parameters that can be used for diagnostic purpose.

Additional blood flow parameters or characteristics may forthcoming that also aid in distinguishing between normal skin and underlying tissue and a disease state, e.g., cancer. Accordingly, the scope of parameters that may be determined using the method and devices of the present methods and devices disclosed herein are not limited to those examples presented here.

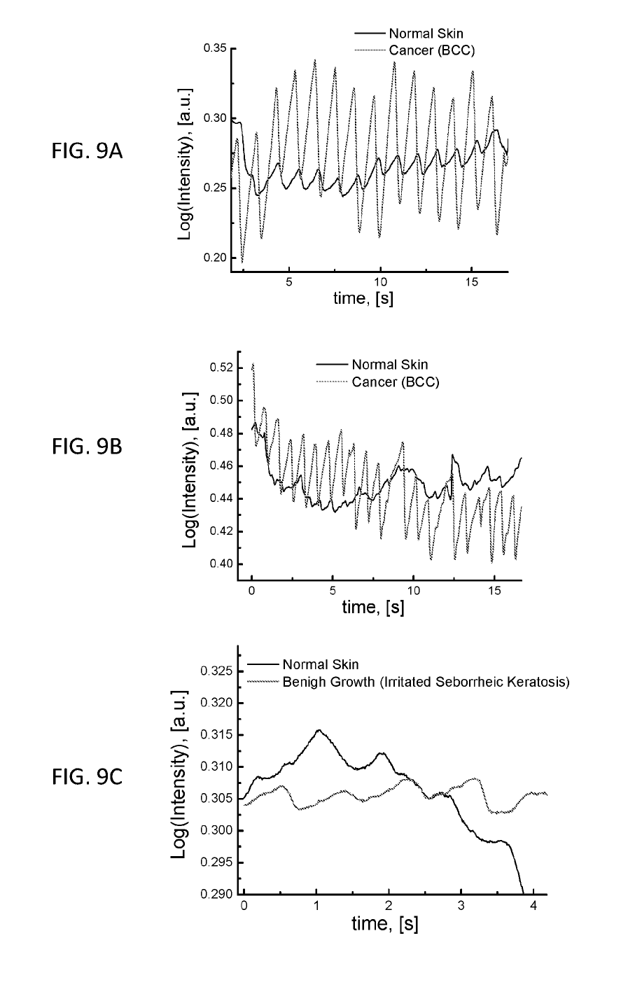

An example of representative data obtained using a device of the present methods and devices disclosed herein and derivation of measured parameters from this data is presented in FIG. 9, taken from a normal (non-disease) skin region, from a skin cancer and from a benign skin condition.

The dynamics of the signal due to periodic action of the heart exhibits pulsative fast and slow rise components. One component may be attributed in part to the removal from the immediate region of chromophores present within the blood. These chromophores absorb, at least in part, the applied photonic energy. When the chromophores in blood, e.g., hemoglobin, are absent, i.e., removed by the blood pressure, more of the applied signal is therefore transmitted via scattering through the skin tissue. When there is more blood in the tissue, less of the signal is transmitted.

In various instances, one or more attributes of a signal obtained from a measured region during the course of these manipulations may not precisely match the pattern of the signal described here. For example, the baseline itself may drift or change over a period of time. In such instances, these variations may be accounted for mathematically to enable direct comparison to other signals obtained elsewhere. Alternatively, these variations may in themselves prove of diagnostic value and therefore be employed in the analysis for determination of a disease state being present. The scope of the present methods and devices disclosed herein therefore is not restricted to data or measured values having the precise shape, forms and magnitudes of those data examples presented here.

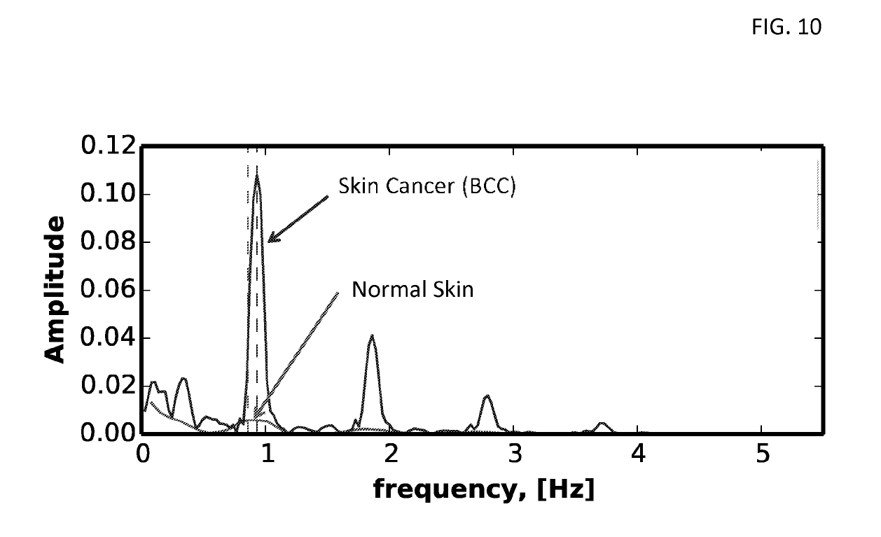

A variety of mathematical tools and approaches may be employed for the analysis of these data. For example, the data may be analyzed using Fourier or Wavelet transforms to determine spectral composition of the signal. One such analysis is shown in FIG. 10 which compares Fourier transforms of the signal from the normal and cancer tissue. These data indicate that the pulsatile component corresponding to the frequency of the heart beat is much more pronounced in the cancer tissue which is consistent with increased blood flow in the malignant phenotype.

The absolute magnitude of the peak amplitude in the spectra (FIG. 10) may be considered to approximately proportional to the absolute volume of chromophores (blood) displaced from the measured region during heat beat.

Other parameters, e.g., maximal signal amplitude, average signal, mean square deviation, higher statistical moments, may be determined using additional mathematical techniques such as signal averaging, filtering and statistical analysis.

The analysis of temporal relationships and correlations between signals from multiple detectors sampling photonic energies at different distances from the light source and at different wavelengths can be used to determine both the lateral and vertical spatial velocities of capillary refill which provides another parameter to selectively characterize flow of blood preferentially along parallel or vertical directions to skin surface.

Once determined, one or more of these parameters may be utilized to assess the likelihood of a disease state in the immediate skin region. Such determinations may be accomplished in a variety of ways. For example, measurements from a suspected skin area may be compared to those of an adjacent area presumed to be healthy or in a non-disease state. If the measures differ by more than a specified amount, then a disease state or probability of a disease state being present may be assigned.

Alternatively, parameters derived from measurements of a suspected skin area may be compared against tabulated values or algorithms obtained through clinical studies examining multiple individuals and lesions. Such comparisons might be performed either electronically or manually. If the values differ by more than a specified amount, then a disease state or probability of a disease state such as melanoma within the measured skin area may be then arrived at.

Alternatively, the present methods and devices disclosed herein can be used for monitoring of suspected area of skin for treatment efficacy assessment.

The scope of the present methods and devices disclosed herein is not constrained to any one form or method of data analysis or determination of probability of disease state.

FIG. 9 presents spectral decomposition of the data from normal skin and a confirmed basal cell carcinoma, BCC. Closer inspection reveals differences between these graphs, however. For instance, it may be noted that the BCC has much stronger amplitude of the pulsatile component which correlates with large amplitude of signal oscillations in FIGS. 9A and 9B. These data clearly demonstrate the ability of measurements obtained with a device of the present methods and devices disclosed herein to usefully distinguish between various skin health states. Through use of various forms of mathematical analyses, the present methods and devices disclosed herein enables determination of disease states in skin regions.

In an alternate example, consider a scenario using a device as disclosed herein whereby blood is being monitored using an photonic energy, e.g., 405 nm, 590 nm or 660 nm light source where excitation and detection areas on the skin are in close proximity. Light penetration is reduced at shorter wavelengths, thereby light at these wavelengths probing dynamics mainly in the capillaries near skin surface. It is a desired feature of the methods and devices disclosed herein to use different wavelengths of light to probe and enable characterization of vascularity at different depths in the tissue.

In alternate embodiments, energies other than photonic energy may be employed, e.g., ultrasound radiation may be used to monitor cutaneous blood dynamics. In one possible embodiment both ultrasound emitter and ultrasound detector are co-located in the inner member; and a time-gated detection system is used to selectively detect cutaneous hemodynamic. In alternate embodiment the light modulated at the ultrasound frequency is absorbed by the blood leading to the emission of ultrasonic radiation at said frequency which is detected by an ultrasound detector co-located next to the light source.

As an alternate form of manipulation of blood flow within a skin region, temperature may be employed. One can readily conceive of device embodiments wherein the use of temperature is employed, to manipulate vascular status and thereby obtain measurements useful for the determination of disease states.

Data analysis using measurements may be performed within the electronics of the device itself, or may be performed in part or in whole upon transference of some or all of the data or mathematical transforms of the data or parameters to one or more data processing units, e.g., laptop computers, internet-based data storage and computing centers, etc. In certain embodiments, measurements can be taken one or more times, e.g., a baseline measurement and one or more measurements.

Such transference of data may be accomplished by wireless, e.g., Bluetooth or WiFi, communication means using appropriately configured electronics within the electronics section 140. Alternatively, wired means, e.g., direct electrical connection between the device and an external device such as a laptop computer, may be employed.

The device itself may present data, parameters, analysis, findings and/or operational status using displays or indicators. Accordingly, displays may utilize alphanumeric characters, simple lights, sounds or other means of conveying information to the user of the device.

Overall, the scope of the present methods and devices disclosed herein is not limited to the examples presented herein. Additional forms of the methods and devices disclosed herein are readily conceivable as well as are forms of the methods and devices disclosed herein involving various combinations of the embodiments presented herein and therefore are within the scope of the methods and devices disclosed herein.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

Devices of the methods and devices disclosed herein may be employed for a variety of uses and applications. Such applications include

### Example 1: Skin Lesion Assessment

In this example, a device as disclosed herein may be employed by a clinician to assess a suspect skin lesion, e.g., a mole-like growth, for characteristics associated with a cancerous state.

The clinician would position the end of the device on a suspected skin lesion, e.g., such that the inner member sensor head was located at the area to be examined. The clinician would then activate the device while maintaining the device against the skin surface. The operational cycle would then automatically occur resulting in measurements being taken automatically by the device. The measurement would then be repeated on the normal-looking skin in the general area of the body where the lesion is located to obtain comparative reference cutaneous blood flow data. Alternatively a second probe would be positioned nearby the lesion and the reference data acquired simultaneously. In another embodiment the probe would contain two independent sensing elements so that the first could be positioned over the lesion and the second over nearby normal tissue to acquire both data sets.

The data from the measurements would be automatically computed and a score indicative of the probability of a cancerous state being present or possibly occurring in the future would then be displayed on the device.

The clinician could then utilize this information to better guide subsequent actions concerning the patient's health, e.g., recommend removal of the lesion by a surgical procedure.

### Example 2: Hand-Held Device for Consumer Use

In this example, a device as disclosed herein may be employed by a consumer to assess a suspect skin lesion, e.g., a mole-like growth, for characteristics associated with a cancerous state.

The consumer would position the end of the device on a suspected skin lesion, e.g., such that the sensor head was located at the area to be examined. The consumer would then activate the device while maintaining the device against the skin surface. The operational cycle would then automatically occur resulting in measurements being taken automatically by the device. The measurement would then be repeated on normal-looking skin in the general area of the body where the lesion is located to obtain reference data. Alternatively a second probe would be positioned nearby the lesion and the reference data acquired simultaneously. In another embodiment the probe would contain two independent sensing elements so that the first could be positioned over the lesion and the second over nearby normal tissue to acquire both data sets.

The data from the measurements would be automatically computed and a score indicative of the probability of a cancerous state being present or possibly occurring in the future would then be displayed on the device.

The consumer could then utilize this information to determine if the consumer should contact a clinician for further assessment. The clinician can then better guide subsequent actions concerning the patient's health, e.g., recommend removal of the lesion by a surgical procedure.

Additional applications and uses of the methods and devices disclosed herein are conceivable and therefore the scope of possible applications is not limited to those examples presented above.

### REFERENCES

1 Dudewicz, E. J. & Van Der Meulen, E. C. Entropy-Based Tests of Uniformity. Journal of the American Statistical Association 76, 967-974, doi:10.1080/01621459.1981. Ser. No. 10/477,750 (1981).
2 Khan, F., Patterson, D., Belch, J. J., Hirata, K. & Lang, C. C. Relationship between peripheral and coronary function using laser Doppler imaging and transthoracic echocardiography. Clin Sci (Lond) 115, 295-300, doi:10.1042/CS20070431 (2008).
3 Cracowski, J. L., Minson, C. T., Salvat-Melis, M. & Halliwill, J. R. Methodological issues in the assessment of skin microvascular endothelial function in humans. Trends Pharmacol Sci 27, 503-508, doi:10.1016/j.tips.2006.07.008 (2006).
4 Holowatz, L. A. & Kenney, W. L. Local ascorbate administration augments NO- and non-NO-dependent reflex cutaneous vasodilation in hypertensive humans. Am J Physiol Heart Circ Physiol 293, H1090-1096, doi:10.1152/ajpheart.00295.2007 (2007).
5 Rousseau, P. et al. Axon-reflex cutaneous vasodilatation is impaired in type 2 diabetic patients receiving chronic low-dose aspirin. Microvasc Res 78, 218-223, doi:10.1016/j.mvr.2009.06.005 (2009).
6 Struijker-Boudier, H. A. et al. Evaluation of the microcirculation in hypertension and cardiovascular disease. Eur Heart J 28, 2834-2840, doi:10.1093/eurheartj/ehm448 (2007).
7 Turner, J., Belch, J. J. & Khan, F. Current concepts in assessment of microvascular endothelial function using laser Doppler imaging and iontophoresis. Trends in cardiovascular medicine 18, 109-116, doi:10.1016/j.tcm.2008.02.001 (2008).
8 Gokce, N. Clinical assessment of endothelial function: ready for prime time? Circulation. Cardiovascular imaging 4, 348-350, doi:10.1161/CIRCIMAGING.111.966218 (2011).
9 Holowatz, L. A., Thompson-Torgerson, C. S. & Kenney, W. L. The human cutaneous circulation as a model of generalized microvascular function. Journal of applied physiology 105, 370-372, doi:10.1152/japplphysiol.00858.2007 (2008).
10 Holowatz, L. A., Santhanam, L., Webb, A., Berkowitz, D. E. & Kenney, W. L. Oral atorvastatin therapy restores cutaneous microvascular function by decreasing arginase activity in hypercholesterolaemic humans. J Physiol 589, 2093-2103, doi:10.1113/jphysiol.2010.203935 (2011).
11 Khalil, Z., LoGiudice, D., Khodr, B., Maruff, P. & Masters, C. Impaired peripheral endothelial microvascular responsiveness in Alzheimer's disease. Journal of Alzheimer's disease: JAD 11, 25-32 (2007).
12 Ming, Z., Siivola, J., Pietikainen, S., Narhi, M. & Hanninen, O. Postoperative relieve of abnormal vasoregulation in carpal tunnel syndrome. Clinical neurology and neurosurgery 109, 413-417, doi:10.1016/j.clineuro.2007.02.014 (2007).
13 Israel, A. K. et al. Peripheral endothelial dysfunction in patients suffering from acute schizophrenia: a potential marker for cardiovascular morbidity? Schizophr Res 128, 44-50, doi:10.1016/j.schres.2011.02.007 (2011).
14 Hogas, S. M. et al. Methods and potential biomarkers for the evaluation of endothelial dysfunction in chronic kidney disease: a critical approach. Journal of the American Society of Hypertension: JASH 4, 116-127, doi:10.1016/j.jash.2010.03.008 (2010).
15 Sokolnicki, L. A., Roberts, S. K., Wilkins, B. W., Basu, A. & Charkoudian, N. Contribution of nitric oxide to cutaneous microvascular dilation in individuals with type 2 diabetes mellitus. American journal of physiology. Endocrinology and metabolism 292, E314-318, doi:10.1152/ajpendo.00365.2006 (2007).
16 Rossi, M., Carpi, A., Galetta, F., Franzoni, F. & Santoro, G. The investigation of skin blood flowmotion: a new approach to study the microcirculatory impairment in vascular diseases? Biomedicine & pharmacotherapy=Biomedecine & pharmacotherapie 60, 437-442, doi:10.1016/j.biopha.2006.07.012 (2006).
17 Green, D. J. et al. Impaired skin blood flow response to environmental heating in chronic heart failure. Eur Heart J 27, 338-343, doi:10.1093/eurheartj/ehi655 (2006).
18 Roustit, M., Simmons, G. H., Carpentier, P. & Cracowski, J. L. Abnormal digital neurovascular response to local heating in systemic sclerosis. Rheumatology (Oxford) 47, 860-864, doi:10.1093/rheumatology/ken065 (2008).
19 Rossi, M. et al. Skin vasodilator function and vasomotion in patients with morbid obesity: effects of gastric bypass surgery. Obes Surg 21, 87-94, doi:10.1007/s11695-010-0286-9 (2011).
20 Holowatz, L. A., Thompson, C. S. & Kenney, W. L. L-Arginine supplementation or arginase inhibition augments reflex cutaneous vasodilatation in aged human skin. J Physiol 574, 573-581, doi:10.1113/jphysiol.2006.108993 (2006).
21 Thompson-Torgerson, C. S., Holowatz, L. A., Flavahan, N. A. & Kenney, W. L. Rho kinase-mediated local cold-induced cutaneous vasoconstriction is augmented in aged human skin. Am J Physiol Heart Circ Physiol 293, H30-36, doi:10.1152/ajpheart.00152.2007 (2007).
22 Trzepizur, W. et al. Microvascular endothelial function in obstructive sleep apnea: Impact of continuous positive airway pressure and mandibular advancement. Sleep medicine 10, 746-752, doi:10.1016/j.sleep.2008.06.013 (2009).
23 Spronk, P. E Zandstra, D. F. & Ince, C. Bench-to-bedside review: sepsis is a disease of the microcirculation. Crit Care 8, 462-468, doi:10.1186/cc2894 (2004).
24 Alba-Alejandre, I., Hiedl, S. & Genzel-Boroviczeny, O. Microcirculatory changes in term newborns with suspected infection: an observational prospective study. International journal of pediatrics 2013, 768784, doi:10.1155/2013/768784 (2013).
25 Takkin, L., Sample, R., Moore, P. & P., G. Prediction of Wound Healing Outcome using Skin Perfusion Pressure & Transcutaneous Oximetry. Wounds 21 (2009).
26 Shapiro, J. & Nouvong, A. in Topics in the Prevention, Treatment and Complications of Type 2 Diabetes (ed Mark Zimering) (InTech, 2011).
27 Carmeliet, P. & Jain, R. K. Angiogenesis in cancer and other diseases. Nature 407, 249-257, doi:10.1038/35025220 (2000).
28 Nishida, N., Yano, H., Nishida, T., Kamura, T. & Kojiro, M. Angiogenesis in cancer. Vascular health and risk management 2, 213-219 (2006).
29 Folkman, J. Angiogenesis in cancer, vascular, rheumatoid and other disease. Nat Med 1, 27-31 (1995).
30 Emmett, M. S., Dewing, D. & Pritchard-Jones, R. O. Angiogenesis and melanoma—from basic science to clinical trials. American journal of cancer research 1, 852-868 (2011).
31 Li, C. Y. et al. Initial stages of tumor cell-induced angiogenesis: evaluation via skin window chambers in rodent models. Journal of the National Cancer Institute 92, 143-147 (2000).
32 Velasco, P. & Lange-Asschenfeldt, B. Dermatological aspects of angiogenesis. The British journal of dermatology 147, 841-852 (2002).
33 Chin, C. W., Foss, A. J., Stevens, A. & Lowe, J. Differences in the vascular patterns of basal and squamous cell skin carcinomas explain their differences in clinical behaviour. The Journal of pathology 200, 308-313, doi:10.1002/path.1363 (2003).
34 Srivastava, A., Laidler, P., Davies, R. P., Horgan, K. & Hughes, L. E. The prognostic significance of tumor vascularity in intermediate-thickness (0.76-4.0 mm thick) skin melanoma. A quantitative histologic study. Am J Pathol 133, 419-423 (1988).
35 Kashani-Sabet, M., Sagebiel, R. W., Ferreira, C. M., Nosrati, M. & Miller, J. R., 3rd. Tumor vascularity in the prognostic assessment of primary cutaneous melanoma. J Clin Oncol 20, 1826-1831 (2002).
36 Toth-Jakatics, R., Jimi, S., Takebayashi, S. & Kawamoto, N. Cutaneous malignant melanoma: correlation between neovascularization and peritumor accumulation of mast cells overexpressing vascular endothelial growth factor. Human pathology 31, 955-960 (2000).
37 Barnhill, R. L., Fandrey, K., Levy, M. A., Mihm, M. C., Jr. & Hyman, B. Angiogenesis and tumor progression of melanoma. Quantification of vascularity in melanocytic nevi and cutaneous malignant melanoma. Lab Invest 67, 331-337 (1992).
38 Weidner, N. Intratumor microvessel density as a prognostic factor in cancer. Am J Pathol 147, 9-19 (1995).
39 Straume, O., Salvesen, H. B. & Akslen, L. A. Angiogenesis is prognostically important in vertical growth phase melanomas. Int J Oncol 15, 595-599 (1999).
40 Ria, R. et al. Angiogenesis and progression in human melanoma. Dermatology research and practice 2010, 185687, doi:10.1155/2010/185687 (2010).
41 Stucker, M. et al. High-resolution laser Doppler perfusion imaging aids in differentiating between benign and malignant melanocytic skin tumours. Acta dermato-venereologica 82, 25-29 (2002).
42 Jain, R. K. Determinants of tumor blood flow: a review. Cancer Res 48, 2641-2658 (1988).
43 Simonsen, T. G., Gaustad, J. V., Leinaas, M. N. & Rofstad, E. K. High interstitial fluid pressure is associated with tumor-line specific vascular abnormalities in human melanoma xenografts. PLoS One 7, e40006, doi:10.1371/journal.pone.0040006 (2012).
44 Lunt, S. J., Fyles, A., Hill, R. P. & Milosevic, M. Interstitial fluid pressure in tumors: therapeutic barrier and biomarker of angiogenesis. Future oncology 4, 793-802, doi:10.2217/14796694.4.6.793 (2008).
45 Heldin, C. H., Rubin, K., Pietras, K. & Ostman, A. High interstitial fluid pressure—an obstacle in cancer therapy. Nat Rev Cancer 4, 806-813, doi:10.1038/nrc1456 (2004).
46 Less, J. R., Posner, M. C., Skalak, T. C., Wolmark, N. & Jain, R. K. Geometric resistance and microvascular network architecture of human colorectal carcinoma. Microcirculation 4, 25-33 (1997).
47 Sevick, E. M. & Jain, R. K. Geometric resistance to blood flow in solid tumors perfused ex vivo: effects of tumor size and perfusion pressure. Cancer Res 49, 3506-3512 (1989).
48 Gaustad, J. V., Simonsen, T. G., Brurberg, K. G., Huuse, E. M. & Rofstad, E. K. Blood supply in melanoma xenografts is governed by the morphology of the supplying arteries. Neoplasia 11, 277-285 (2009).
49 Lyng, H., Skretting, A. & Rofstad, E. K. Blood flow in six human melanoma xenograft lines with different growth characteristics. Cancer Res 52, 584-592 (1992).
50 Sensky, P. L., Prise, V. E., Tozer, G. M., Shaffi, K. M. & Hirst, D. G. Resistance to flow through tissue-isolated transplanted rat tumours located in two different sites. British journal of cancer 67, 1337-1341 (1993).
51 Baish, J. W. & Jain, R. K. Fractals and cancer. Cancer Res 60, 3683-3688 (2000).
52 Gessner, R. C., Aylward, S. R. & Dayton, P. A. Mapping microvasculature with acoustic angiography yields quantifiable differences between healthy and tumor-bearing tissue volumes in a rodent model. Radiology 264, 733-740, doi:10.1148/radiol.12112000 (2012).
53 Garry, A. et al. Cellular mechanisms underlying cutaneous pressure-induced vasodilation: in vivo involvement of potassium channels. Am J Physiol Heart Circ Physiol 289, H174-180, doi:10.1152/ajpheart.01020.2004 (2005).
54 Abraham, P., Fromy, B., Merzeau, S., Jardel, A. & Saumet, J. L. Dynamics of local pressure-induced cutaneous vasodilation in the human hand. Microvasc Res 61, 122-129, doi:10.1006/mvre.2000.2290 (2001).
55 Fromy, B. et al. Early decrease of skin blood flow in response to locally applied pressure in diabetic subjects. Diabetes 51, 1214-1217 (2002).
56 Livesey, A. K. & Brochon, J. C. Analyzing the distribution of decay constants in pulse-fluorimetry using the maximum entropy method. Biophys J 52, 693-706 (1987).
57 Jacques, S. L. Optical properties of biological tissues: a review. Physics in medicine and biology 58, R37-61, doi:10.1088/0031-9155/58/11/R37 (2013).

What is claimed is:

1. A device for measuring cutaneous blood circulation in a region between two or more areas of the skin, the device comprising a sensor comprising a localized discrete photonic excitation source which illuminates the first area of the skin and a photonic detector which detects light from another area of the skin, wherein the sensor is configured to measure blood flow dynamics and to determine one or more blood flow parameters within the said region of the skin.

2. The device of claim 1, wherein the device measures spatially resolved cutaneous blood microcirculation by determining one or more blood flow parameters in a spatially resolved manner.

3. The device of claim 1, wherein the photonic detector measures an applied photonic energy absorption by a component of blood.

4. The device of claim 1, wherein the photonic detector has an active area of less than 1 mm², between 1 mm² and 5 mm² and more than 5 mm².

5. The device of claim 1, wherein the photonic excitation source has an active area of less than 1 mm², between 1 mm² and 5 mm² and more than 5 mm².

6. The device of claim 1, wherein one or more discrete photonic detectors are located at less than 0.5 mm, 0.5 mm to 2 mm, 2 mm to 5 mm, or more than 5 mm distance from the localized discrete excitation source.

7. The device of claim 1, wherein the photonic detector is an imaging detector.

8. The device of claim 1, wherein photonic energy is delivered to and collected from one or more areas of the skin region using optical fibers.

9. The device of claim 1, wherein the sensor comprises a plurality of photonic detectors, wherein each receiver for a photonic detector is located at different distances from the emission location of the photonic excitation source of the sensor.

10. The device of claim 1, wherein the device is configured to measure one or more blood flow parameters within an area of the skin region, wherein the area is between about 0.1 mm and 1 mm, between 1 and 2 mm, between 2 mm and 5 mm, and more than 5 mm in size.

11. The device of claim 1, wherein the photonic excitation source emits light at wavelengths below 400 nm, between 400 nm and 450 nm, between 450 nm and 500 nm, between 500 nm and 550 nm, between 550 nm and 600 nm, between 600 nm and 650 nm, between 650 nm and 700 nm, or above 700 nm.

12. The device of claim 1, wherein the sensing member comprises a convex, concave or non-planar surface for improved contact with the skin.

13. The device of claim 1, wherein a device contains more than one sensor which perform measurement simultaneously on more than one location of skin.

14. The device of claim 1, wherein the excitation source is ultrasound source and the photonic detector is an ultrasound detector.

15. The device of claim 14, wherein the ultrasound detector is an ultrasound imaging detector.

16. A method to detect a change in cutaneous blood circulation, comprising:
a) providing one or more localized, discrete photonic excitation sources to illuminate the first area of the skin and one or more localized, discrete photonic detectors to detect light emanating from the second area of the skin to measure blood flow dynamics and determine one or more blood flow parameters in a spatially resolved manner in a region located between the first and the second area of the skin;
b) analyzing and quantifying the one or more measured blood flow parameters from said one or more regions of the skin;
c) assessing said blood flow parameters to identify blood flow; and
d) comparing the blood flow to one or more other assessments to determine the presence of a disease state.

17. The method of claim 16, wherein the skin area, illuminated by the excitation source and the skin area from which light is monitored, are less than 1 mm², 1 mm² to 5 mm², and more than 5 mm² in size.

18. The method of claim 16, wherein multiple localized discrete photonic detectors are used to detect light from multiples discrete skin areas surrounding the skin area illuminated by the photonic excitation source.

19. The method of claim 16, wherein the skin area illuminated by the localized excitation source and the skin area(s) from which light is monitored are located at less than 0.5 mm, 0.5 mm to 2 mm, 2 mm to 5 mm, or more than 5 mm distance from each other.

20. The method of claim 16, wherein the photonic detector is an imaging detector used to detect light from the areas surrounding the area illuminated by the photonic excitation source.

21. The method of claim 16, wherein the disease state is cancer.

22. The method of claim 16, wherein the cancer is skin cancer.

23. The method of claim 16, wherein the cancer is benign or malignant.

24. The method of claim 16, wherein the cancer is metastatic.

25. The method of claim 16, wherein the disease state is hypercholesterolemia, Alzheimer disease, carpal tunnel syndrome, schizophrenia, hypertension, renal disease, type 2 diabetes, peripheral vascular disease, atherosclerotic coronary artery disease, heart failure, systemic sclerosis, obesity, primary aging, sleep apnea, neonatal & adult sepsis, wound healing, or a combination thereof.

26. The method of claim 16, wherein the blood flow parameters are analyzed and quantified.

27. The method of claim 16, wherein analyzing the one or more measured blood flow parameters comprises utilizing at least one of Fourier transform, wavelet transform, digital filtering, time correlation and statistical analysis of time series.

28. The method of claim 16, wherein assessing blood flow parameters relative to one or more other assessments comprises comparing signal frequencies, amplitudes and spectra, amplitude distributions obtained from the skin region with a reference skin region.

29. The method of claims 16 and 20-21, wherein the skin region is within a lesion suspicious for cancer.

30. The method of claims 16 and 20-21, wherein the reference skin region does not include cancer.

31. The method of claim 16, wherein analyzing the one or more measured blood flow parameters comprises determining temporal relationships and correlations between signals acquired from a plurality of photonic detectors, where each receiver for a photonic detector is located at a different distance from the emission of the photonic excitation source.

32. The method of claim 16, wherein analyzing the one or more measured blood flow parameters comprises determining temporal relationships and correlations between signals acquired from a plurality of photonic detectors at different wavelengths emitted from the photonic excitation source.

33. The method of claim 16, further comprising performing a hemodynamic analyses on a plurality of skin region locations, wherein the hemodynamic analysis of each location is compared to another location to determine or compare disease status.

34. The method of claim 16, wherein the one or more blood flow parameters provides a hemodynamic profile of the skin region, wherein relative amount of blood flow is determined from the shape of pulsatile hemodynamic profile, and wherein the extent of blood flow is indicative of the presence of the disease state.

35. The method of claim 16, wherein the excitation source is ultrasound source and the photonic detector is an ultrasound detector.

36. The method of claim 35, wherein the ultrasound detector is an ultrasound imaging detector.
